(19) United States
(12) Patent Application Publication
(10) Pub. No.: US 2026/0091128 A1
(43) Pub. Date: Apr. 2, 2026

(54) POLYION COMPLEXES FOR BIOMOLECULAR DELIVERY

(71) Applicants: THE UNIVERSITY OF NORTH CAROLINA AT CHAPEL HILL, Chapel Hill, NC (US); TECHNISCHE UNIVERSITAET DRESDEN, Dresden (DE)

(72) Inventors: Alexander Kabanov, Chapel Hill, NC (US); Jacob Daggett Ramsey, Chapel Hill, NC (US); Konstantin Lukianov, Chapel Hill, NC (US); Kyoungtea Kim, Chapel Hill, NC (US); Duhyeong Hwang, Chapel Hill, NC (US); Rainer Jordan, Freital (DE)

(21) Appl. No.: 19/112,168

(22) PCT Filed: Sep. 15, 2023
(86) PCT No.: PCT/US2023/032942

§ 371 Date: Mar. 14, 2025

Related U.S. Application Data
(60) Provisional application No. 63/407,330, filed on Sep. 16, 2022.

(51) Int. Cl. A61K 47/69; A61K 47/54; B82Y 5/00; C08G 73/02; C08G 73/08

(52) U.S. Cl. CPC A61K 47/6935; A61K 47/549; C08G 73/0233; C08G 73/08; B82Y 5/00

(57) ABSTRACT

In one aspect, a block copolymer described herein comprises a hydrophilic block including oxazoline monomer or oxazine monomer, and a cationic block comprising monomer including a linear or branched polyamine side chain. In another aspect, a polyion complex comprises a block copolymer comprising a hydrophilic block including oxazoline monomer or oxazine monomer, and a cationic block comprising monomer including a linear or branched cationic polyamine side chain, and a negatively charged biomolecular species associated with the block copolymer. The negatively charged biomolecular species can comprise one or more nucleic acids, such as RNA, DNA, and/or other oligonucleotides.

### RELATED APPLICATION DATA

The present application claims priority pursuant to Article 8 of the Patent Cooperation Treaty to U.S. Provisional Patent Application Ser. No. 63/407,330 filed Sep. 16, 2022, which is incorporated herein by reference in its entirety.

### FIELD

The present invention relates to polyion complexes and, in particular, to polyion complexes comprising cationic block copolymers. Such complexes can be free of lipids and/or PEI-PEG copolymer.

### BACKGROUND

Despite dramatic progress in application of the lipid nanoparticles (LNP) in mRNA vaccines, the gene delivery systems face challenging barriers such as off-target immune responses and low delivery efficiency. Genetic material, such as plasmid DNA (pDNA), is challenging to deliver in vivo due to degradation by DNases, inefficient delivery into the cell, and lysosomal entrapment and degradation. Polymer-mediated gene delivery focuses on combining cationic polymers with negatively charged genetic material to form polyion complexes (polyplexes). One of the very well-studied cationic polymers used for plasmid delivery is a block copolymer of polyethyleneimine (PEI) and polyethylene glycol (PEG). Though PEI-PEG based polyplexes have a high transfection efficiency, the high molecular weight of PEI is required for efficient transfection, which makes these polyplexes cytotoxic and unsuitable for in vivo application. The addition of PEG results in a beneficial “stealth” effect allowing for enhanced circulation in vivo. Due to its relative inertness and “stealth” property, PEG quickly became used in many cancer treatments, such as in breast and ovarian cancer drugs. However, the ubiquity of PEG is problematic in causing the rise of PEG-antibodies, which decreases the efficacy of life-saving PEG-based treatments. A recent study reports that 72% of individuals have detectable levels of PEG antibodies, which is driving a significant need for alternative polymers that also employ stealth properties.

### SUMMARY

In view of the foregoing disadvantages, cationic block copolymers and associated polyion complexes are described herein. In some embodiments, the polyion complexes can be modified for the targeted delivery of nucleic acids and/or oligonucleotides to various cellular species.

In one aspect, a block copolymer described herein comprises a hydrophilic block including oxazoline monomer or oxazine monomer, and a cationic block comprising monomer including a linear or branched polyamine side chain. In some embodiments, the hydrophilic block is non-ionic. For example, the hydrophilic block can be of the formula

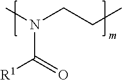

wherein R¹ is selected from the group consisting of alkyl and cycloalkyl, each optionally substituted with hydroxyl, —SH, and C(O)OR², wherein R² is selected from the group consisting of hydrogen and alkyl, and wherein m ranges from 10-500.

As described herein, the block copolymer also comprises a cationic block including monomer comprising a linear or branched polyamine side chain. In comprising amine groups, the polyamine side chain can be reversibly cationic, depending on protonation of the amines in various pH ranges. In some embodiments, the cationic block is of the formula:

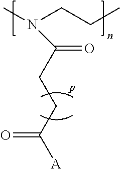

wherein A is the linear or branched or branched polyamine side chain, n ranges from 10-300 and p ranges from 0 to 10. The cationic block, in some embodiments, further comprises non-polyamine monomer.

In some embodiments, the block copolymer can further comprise a third block being more hydrophobic than the hydrophilic block. As described further herein, block copolymer can be provided in a dried film or powder form. The dried film comprising the block copolymer can then be contacted with solution comprising negatively charged biomolecular species to form polyion complexes described herein.

In another aspect, a polyion complex comprises a block copolymer comprising a hydrophilic block including oxazoline monomer or oxazine monomer, and a cationic block comprising monomer including a linear or branched cationic polyamine side chain, and a negatively charged biomolecular species associated with the block copolymer. The negatively charged biomolecular species can comprise one or more nucleic acids, such as RNA, DNA, and/or other oligonucleotides. The nucleic acids can be natural or synthetic. The hydrophilic block and cationic block can have structure described above, in some embodiments. The polyion complex can exhibit a hydrodynamic diameter on the nanometer scale, including a hydrodynamic diameter of 50 nm to 150 nm.

In another aspect, dispersions are described herein. In some embodiments, a dispersion comprises an aqueous or aqueous-based continuous phase, and a dispersed phase comprising polyion complexes, the polyion complexes comprising a block copolymer including a hydrophilic block comprising oxazoline monomer or oxazine monomer, and a cationic block comprising monomer including a linear or branched cationic polyamine side chain, and a negatively charged biomolecular species associated with the block copolymer. As described further herein, the polyion complexes of the dispersion, in some embodiments, have an average hydrodynamic diameter of 10 nm to 300 nm and exhibit a polydispersity index (PDI) of 0.1-0.3. In some embodiments, the PDI can be less than 0.1, such as 0.01-0.09. Additionally, the block copolymer and negatively charged biomolecular species can have structure and composition described above. In some embodiments, the dispersion further comprises an amphiphilic excipient, such as one or more polar lipids. Moreover, the dispersion may further comprise buffer and/or one or more counterion species, such as sulfate anion. In some embodiments, dispersions described herein are isotonic. In such embodiments, the dispersions can include one or more saccharides. The one or more saccharides can be a substitute for sodium chloride in the isotonic composition.

In another aspect, methods of producing dispersions are described herein. A method of forming a dispersion, in some embodiments, comprises providing a dried film comprising a block copolymer, the block copolymer including a hydrophilic block comprising oxazoline monomer or oxazine monomer, and a cationic block comprising monomer including a linear or branched polyamine side chain. The dried film is contacted with an aqueous or aqueous-based continuous phase comprising a negatively charged biomolecular species, thereby forming a dispersed phase of polyion complexes in the continuous phase, the polyion complexes comprising the negatively charged biomolecular species associated with the block copolymer. In some embodiments, the negatively charged biomolecular species comprises one or more nucleic acids, such as DNA, RNA, and/or other oligonucleotides. Moreover, the cationic copolymer can have any composition and/or structure described herein. Additionally, in some embodiments, the dried film further comprises an amphiphilic excipient. The amphiphilic excipient becomes part of the dispersion following contact with the aqueous or aqueous-based continuous phase. In some embodiments, the amphiphilic excipient comprises one or more polar lipids.

These and other embodiments are further described in the following detailed description.

### BRIEF DESCRIPTION OF THE DRAWINGS

FIG. 1 is a schematic depiction of optimization strategy for a targeted poly(2-oxazoline)-based polymer capable of transfecting immune cells, such as macrophages, via polyplex formation with plasmid DNA. Components varied in polymer design include the following groups: hydrophilic block, cationic block, copper-free or copper-based click method for conjugating mannose targeting moiety, and an optional thermosensitive-hydrophobic group.

FIG. 2A is a synthesis strategy schematic for development of cationic polymers as gene delivery vehicles with a clickable moiety for further modification. A diblock copolymer composed of pMestOx and pEtOx was synthesized via sequential LCROP of 2-oxazolines initiated by p-toluenesulfonic acid methyl ester and terminated with DBCO-amine. Diethylenetriamine (DET) or tris(2-aminoethyl) amine (TREN) was incorporated to the methylester group of the diblock copolymer via ester-amide exchange reaction. Resulting polymers were DMD2, DMT2, or DED2.

FIG. 2B is a synthesis strategy schematic for development of cationic polymers as gene delivery vehicles with a clickable moiety for further modification. Diblock and triblock copolymers composed of pEtOx, pMestOx, and piPrOx (triblock only) were synthesized via sequential LCROP with propargyl p-toluenesulfonate as the initiator and terminated with piperidine (R). DET was incorporated into the methylester group via ester-amide exchange reaction. Polymers were also mannosylated resulting in four final polymers consisting of EtOx, DET, and optional mannose: AED2, MED2, AED3, and MED3.

FIG. 3A is a schematic and a nuclear magnetic resonance (NMR) graphical representation of diblock (AED2) polymers.

FIG. 3B is a schematic and an NMR graphical representation of triblock (AED3) polymers.

FIG. 4A is a schematic and an NMR graphical representation of mannosylated diblock (MED2) polymers. MED2 has 37% mannose conjugation.

FIG. 4B is a schematic and an NMR graphical representation of mannosylated triblock (MED3) polymers. MED3 has 31% mannose conjugation.

FIG. 5A is a graphical representation of physicochemical characterization of cationic moieties, DET and TREN. The 2-(p-toluidino)-6-naphthalene sulfonic acid (TNS) assay results of pMeOx-pMestDET and pMeOx-pMestTREN. Error bars represent ±SD.

FIG. 5B is a graphical representation of physicochemical characterization of cationic moieties, DET and TREN. The pH titration curves of pMeOx-pMestDET solutions are displayed.

FIG. 5C is a graphical representation of physicochemical characterization of cationic moieties, DET and TREN. The pH titration curves of pMeOx-pMestTREN solutions are displayed.

FIG. 6A is a gel imaging representation of physicochemical characterization of cationic moieties, DET and TREN. An image of agarose gel electrophoresis for pDNA complexed with DMD2 (pMeOx-pMestDET, left) and DMT2 (pMeOx-MestTREN, right) at various N/P ratios is shown. pDNA was visualized by ethidium bromide staining. P: pDNA alone.

FIG. 6B is analysis of physicochemical characterization of cationic moieties, DET and TREN. Polyplex size distribution of DMD2-pDNA and DMT2-pDNA complexes at N/P=10 as analyzed by DLS is displayed. Z-average diameter given in nm±SD.

FIG. 7A is a graphical representation of polyplex characterization. Stability of polyplexes by size (bars) and polydispersity index (PDI) (symbols) at N/P 20 after 30 min incubation at room temperature (RT) or after 30 min incubation at room temperature followed by a 60 min incubation at 37° C. in 150 mM NaCl (37° C.+NaCl). ns: not significant.

FIG. 7B is a gel electrophoresis image showing polyplex characterization. Gel electrophoresis showing complexation between polymers and luc-pDNA at NP ratios 5, 10, and 20 after 30 min incubation at RT.

FIG. 7C are a transmission electron microscopy (TEM) images showing polyplex characterization. TEM images of AED2, MED2, AED3, or MED3-based polyplexes prepared at N/P 20 after 30 min incubation at RT.

FIG. 7D is a graphical representation of polyplex characterization. RAW264.7 cell viability after 24 hour treatment with polyplexes prepared at N/P 10 and 20.

FIG. 8 is a gel electrophoresis image representing complexation between polymers AED2, MED2, AED3, MED3 and luc-pDNA at NP ratios 1 and 2 after 30 min incubation at RT. Polyplexes were measured on a 1% agarose gel at 100V for 45 min.

FIG. 9A is a graphical representation of the effect of mannosylation on transfection. An ethidium bromide (EtBr) assay of polyplexes prepared with luc-pDBA and AED2, MED2, AED3, and MED3 at various N/P ratios from 0.1 to 20. EtBr displacement (%) represent amount of EtBr displaced by polymer, or a representation of the binding between the polymer and pDNA. All polyplexes displace majority of EtBr at N/P 2 or higher.

FIG. 9B is a graphical representation of the effect of mannosylation on transfection. Decreasing ratio of mannosylated polymer MED2 compared to ED2 shows antagonistic effect on transfection of RAW264.7 macrophages. Ratio of polymers mixed prior to forming polyplexes at N/P ratio 10.

FIG. 10A is a graphical representation of POx-pDNA polyplex optimization via in vitro transfection of IC21 macrophages. Cells transfected with either DMD2-based (DET) or DMT2-based (TREN) polyplexes at N/P 10 and 20. *p<0.05, **p<0.01, ***p<0.001, ****p<0.0001.

FIG. 10B is a graphical representation of POx-pDNA polyplex optimization via in vitro transfection of IC21 macrophages. Cells transfected with either DMD2-based (MeOx) or DED2-based (EtOx) polyplexes at N/P 10 and 20. *p<0.05, **p<0.01, ***p<0.001, ****p<0.0001.

FIG. 11A is a graphical representation of in vitro transfection screening. Screening macrophage transfection with optimized targeted and untargeted diblock and triblock polymers in RAW264.7 cells. Controls are cells alone, pDNA alone, and GeneJuice, a commercial transfection reagent. ns: not significant. *p<0.05, **p<0.01, ***p<0.001, ****p<0.0001.

FIG. 11B is a graphical representation of in vitro transfection screening. Screening macrophage transfection with optimized targeted and untargeted diblock and triblock polymers in BMDM cells. Controls are cells alone, pDNA alone, and GeneJuice, a commercial transfection reagent. ns: not significant. *p<0.05, **p<0.01, ***p<0.001, ****p<0.0001.

FIG. 12 is a graphical representation of the zeta potential of polyplexes. Polyplexes made with polymers AED2, MED2, AED3, or MED3 and luc-pDNA were prepared at N/P 20 and incubated at RT for 30 min.

FIG. 13 are confocal microscopy images of polyplex uptake in RAW264.7 macrophages. Images taken after 24-hour treatment with polyplexes. Polyplexes were prepared at N/P 20 with controls being Cy5-pDNA alone, and GeneJuice. Cellular compartments such as the cell membrane (WGA-555), lysosomes (LAMP1), and nucleus (DAPI) were stained.

FIG. 14 is a graphical representation of confocal imaging quantification of uptake. RAW264.7 macrophages were treated with polyplexes prepared at N/P 20. Cy5+ signal represents internalized Cy5-pDNA as average signal intensity per nucleus.

FIG. 15 is a graphical representation of transfection in Hela cells induced by polyplexes formed by a dried-film method. TL represents triblock long butyl block, p(EtOx)₃₈-p(BuOx)₂₁-p(Mest-DET)₃₁. TS represents triblock short butyl block, p(EtOx)₃₅-p(BuOx)₁₀-p(Mest-DET)₃₀. CL represents cardiolipin.

FIG. 16 is a graphical representation of transfection in HeLa cells induced by polyplexes freshly made or freeze-dried (lyophilized). TL represents triblock long butyl block, p(EtOx)₃₈-p(BuOx)₂₁-p(Mest-DET)₃₁. TS represents triblock short butyl block, p(EtOx)₃₅-p(BuOx)₁₀-p(Mest-DET)₃₀. CL represents cardiolipin.

FIG. 17 is a graphical representation of the influence of sodium sulphate on polyplex formation. Size distribution by intensity measured by Dynamic Light Scattering (DLS) is shown for solutions TL/mRNA polyplex containing 1 mM (medium grey), 10 mM (light grey), or 100 mM (dark grey) Na₂SO₄.

FIG. 18 is a graphical representation of the influence of buffers on polyplex formation. Size distribution by intensity measured by Dynamic Light Scattering (DLS) is shown for TL/mRNA polyplex solutions containing HEPES (medium grey), TBE (dark grey), or PBS (light grey).

FIG. 19 is a graphical representation of the stability of polyplex in buffered solutions. Size distribution by intensity measured by Dynamic Light Scattering (DLS) is shown for freshly prepared 10-fold diluted TL/mRNA polyplex solution (dark grey) and after storage at room temperature for 3 days (light grey).

FIG. 20 is a graphical representation of the stability of polyplex in buffered solutions. Size distribution by intensity measured by Dynamic Light Scattering (DLS) is shown for freshly prepared TL/mRNA polyplex solution (dark grey) and after its storage at room temperature for 1 day (light grey).

FIG. 21 is a graphical representation of the influence of NaCl on polyplex formation. Size distribution by intensity measured by Dynamic Light Scattering (DLS) is shown for TL/mRNA polyplex solution in 1 mM Na₂SO₄, 1 mM HEPES (dark grey) and after addition to this sample 154 mM NaCl (light grey).

FIG. 22 is a graphical representation of polyplexes formed in a glucose-based isotonic solution. Size distribution by intensity measured by Dynamic Light Scattering (DLS) is shown for freshly prepared TL/mRNA polyplex solution containing 0.2 mg/ml mRNA, 1 mM Na₂SO₄, 1 mM HEPES, 5% glucose (dark grey) and after its storage at room temperature for 1 day (light grey).

FIG. 23 is an agarose gel electrophoresis demonstration of mRNA and TL/mRNA polyplexes at different N/P ratios. N/P ratios are designated above the lanes. The left lane: Thermo Scientific™ RiboRuler High Range RNA Ladder

FIG. 24 is a graphical representation of polyplexes formed with unmodified mRNA (top left panel) or N1-methyl-pseudouridine-mRNA (m1Ψ-mRNA, bottom left panel). Size distribution by intensity measured by Dynamic Light Scattering (DLS) is shown for freshly prepared TL/mRNA polyplex solution containing 0.2 mg/ml mRNA or m1Ψ-mRNA, 1 mM Na₂SO₄, 1 mM HEPES, 5% glucose. Medium grey lines—N/P=3, light grey lines—N/P=10, dark grey lines—N/P=20, black lines—N/P=10, 1 mM MgCl₂. The table on the right shows characteristic sizes and dispersity of the polyplex particles.

FIG. 25 is a graphical representation of polyplexes formed in the presence of cardiolipin 16:1. Size distribution by intensity measured by Dynamic Light Scattering (DLS) is shown for freshly prepared polyplex solution containing 0.2 mg/ml m1Ψ-mRNA, 1 mM Na₂SO₄, 1 mM HEPES, 1 mM MgCl₂, 5% glucose. Dark grey lines—TL films; light grey lines—TL plus cardiolipin films. Top panel—N/P=10, bottom panel—N/P=15.

FIG. 26 is a graphical representation of a comparison of freshly prepared and lyophilized-rehydrated polyplexes formed by unmodified mRNA. Size distribution by intensity measured by Dynamic Light Scattering (DLS) is shown, dark grey lines—samples before lyophilization, light grey lines—samples after lyophilization. All samples contained 0.2 mg/ml mRNA, 1 mM Na₂SO₄, 1 mM HEPES, 5% glucose. N/P ratios for TL polymer, the presence of 1 mM MgCl₂ or cardiolipin 16:1 are designated on the panels.

FIG. 27 is a graphical representation of a comparison of freshly prepared and lyophilized-rehydrated polyplexes formed by m1Ψ-mRNA. Size distribution by intensity measured by Dynamic Light Scattering (DLS) is shown, dark grey lines—samples before lyophilization, light grey lines—samples after lyophilization. All samples contained 0.2 mg/ml m1Ψ-mRNA, 1 mM Na₂SO₄, 1 mM HEPES, 5% glucose. N/P ratios for TL polymer, the presence of 1 mM MgCl₂ or cardiolipin 16:1 are designated on the panels.

FIG. 28 illustrates a method of forming a dispersion according to some embodiments described herein.

FIG. 29 illustrates block copolymers of various structures according to some embodiments.

FIGS. 30A-30C provide hydrodynamic diameters and PDI of polyion complexes formed via a dry film method described herein.

FIGS. 31A-31B provide hydrodynamic diameters and PDI of polyion complexes formed via a dry film method described herein.

FIGS. 32A-32C provide hydrodynamic diameters and PDI of polyion complexes formed via a dry film method described herein.

FIGS. 33A-33B provide hydrodynamic diameters and PDI of polyion complexes formed via a dry film method described herein.

FIGS. 34A-34B provide hydrodynamic diameters and PDI of polyion complexes formed via a dry film method described herein.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### I. Cationic Block Copolymers

In one aspect, a block copolymer described herein comprises a hydrophilic block including oxazoline monomer or oxazine monomer, and a cationic block comprising monomer including a linear or branched polyamine side chain. In some embodiments, the hydrophilic block is non-ionic. For example, the hydrophilic block can be of the formula

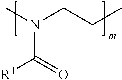

wherein R¹ is selected from the group consisting of alkyl and cycloalkyl, each optionally substituted with hydroxyl, —SH, and C(O)OR², wherein R² is selected from the group consisting of hydrogen and alkyl, and wherein m ranges from 10-300. For example, in some embodiments, R¹ is alkyl such as methyl (2-methyl-2-oxazoline, MeOx) or ethyl (2-ethyl-2-oxazoline, EtOx). Length of the hydrophilic block can be varied according to desired structure of the block copolymer. In some embodiments, m ranges from 10-500, 20-400, 10-300, 20 to 80 or 30 to 70. As described herein, the hydrophilic block can alternatively be formed of oxazine monomer. Oxazine monomer can employ the same R¹ functionality as the oxazoline monomer, in some embodiments.

The block copolymer also comprises a cationic block including monomer comprising a linear or branched polyamine side chain. In comprising amine groups, the polyamine side chain can be reversibly cationic, depending on protonation of the amines in various pH ranges. The polyamine side chain, in some embodiments, comprises at least 3 amine groups. For example, the polyamine side chain can comprise 3-10 amine groups. The number and configuration of amines of the block copolymer can determine how the block copolymer will interact with or incorporate negatively charged biomolecular species, such as nucleic acids of various structure and charge, when forming polyion complexes. Varying the arrangement of amines and structure of cationic side chains can yield improvements, including enhanced condensation of nucleic acid cargo.

The type of amine also determines the pKa which can impact the level of protonation of the polyion complex when the complex reaches the endosome during transfection. Primary amines, for example, are more protonated at endosomal pH compared to secondary and tertiary amines. In some embodiments, the reversibly cationic amine groups in the side chain exhibit an effective or average pKa from about 4 to about 11, form about 4 to about 10, or from about 6 to about 9. The amine groups in the side chain can exhibit differing pKa values based on position in the side chain. The first amine moiety of the chain closest to the copolymer backbone, for example, can have a pKa of 4 to 7 or 4 to 6, while the second amine moiety in the chain can have a pKa of 6 to 11 or 7 to 9. Such structure and properties can permit copolymers described herein to display buffering capacity in both acidic and alkaline regions. The methyl-based DET-containing copolymer described further herein displays buffering capacity in both acidic and alkali areas with effective pKa values of about 6.0 and 11.0, while the TREN-containing polymer displayed a buffering capacity in the ranges corresponding to effective pKa of about 4.0. Moreover, ethyl-based DET-containing copolymer described further herein displayed a buffering capacity in both acidic and alkali regions with effective pKa close to 4.3-8.8 respectively. pKa of pEtOx-pMestDET, for example, is at approximately pH 4.3 and 8.8.

In some embodiments, the cationic block is of the formula:

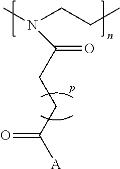

wherein A is the linear or branched or branched polyamine side chain, n ranges from 10-300, 20 to 80, or 30 to 70, and p ranges from 0 to 10. In some embodiments, the cationic polyamine side chain is constructed using diethylenetriamine (DET, linear) or tris(2-aminoethyl) amine (TREN, branched). As described herein, the value of n can be varied to achieve the desired block copolymer structure and cationic charge for polyion complex formation. In some embodiments, the specific identity of the branched polyamine side chain and/or the value of n are selected to achieve a desired N/P ratio of the polyion complex. The N/P ratio is the ratio of positively charged amines on the cationic block copolymer to the negatively charged phosphates of the nucleic acid or oligonucleotide. For the positively charged amines, only the first amine with the lowest pKa and any amine having a pKa within 1 of the first amine are included in the N/P calculation. A polyion complex can exhibit a N/P ratio of 1 to 20, in some embodiments. As described further herein, one or more polar lipids may be associated with or part of the polyion complex. In such embodiments, negatively charged phosphates of the lipid(s) are included in the N/P ratio. In some embodiments, a polyion complex comprising one or more lipids has an N/P ratio of 10-25 or 15-20.

The block copolymer, in some embodiments, further comprises a third block being more hydrophobic than the hydrophilic block. The hydrophobic block can have any composition consistent with the objectives described herein. In some embodiments, the third block comprises oxazoline monomer or oxazine monomer having comprising a propyl, isopropyl, or butyl side chain. Length of third block can be varied to achieve the desired block copolymer structure for polyion formation. In some embodiments, q of the third block has a value ranging from 10-300, 20 to 80, or 30 to 70. When the hydrophobic third block is present, the block copolymer can exhibit a ABC structure or ACB structure, wherein A is the hydrophilic block, B is the hydrophobic block, and C is the cationic block.

The cationic block, in some embodiments, further comprises non-polyamine monomer. The non-polyamine monomer can comprise oxazoline monomer or oxazine monomer having comprising a propyl or butyl side chain. The non-polyamine monomer and monomer including the polyamine side chain have a periodic distribution in the cationic block. Alternatively, the non-polyamine monomer and monomer including the polyamine side chain have a random distribution in the cationic block.

The block copolymer, in some embodiments, can be terminated in a click chemistry moiety for subsequent functionalization with a targeting ligand. The block copolymer, for example, can be terminated in a click chemistry moiety selected from the group consisting of BCN, DBCO, TCO, tetrazine, alkyne, and azide. The targeting ligand, in some embodiments, is selected from receptor ligands, carbohydrate moieties, peptides, and antibodies. Specific identity of the targeting ligand is dependent on the desired cellular environment for locating the polyion complex for transfection. For example, the targeting strategy can be used to deliver nucleic acids to immune cells via the polyion complexes described herein. Therefore, ligands or moieties targeting monocytes and/or macrophages can be employed with block copolymers described herein.

The block copolymer, in some embodiments, is provided in a dried film or powder form, including lyophilized powder. As described further herein, the dried film or powder can also include one or more excipients, such as polar lipid. The dried film or powder can be contacted with solution comprising negatively charged biomolecular species, such as nucleic acids or oligonucleotides, to form polyion complexes having composition and/or properties described herein. In some embodiments, the dried film or powder can be coated on or otherwise associated with one or more supports or carriers. Supports or carriers can include particles, such as plastic, glass, or silica particles. The supports or carriers can be porous or non-porous. Accordingly, films or powders comprising block copolymers described herein can remain in storage until combined with the desired biomolecular solution for polyion complex formation.

### II. Polyion Complexes

In another aspect, a polyion complex comprises a block copolymer comprising a hydrophilic block including oxazoline monomer or oxazine monomer, and a cationic block comprising monomer including a linear or branched cationic polyamine side chain, and a negatively charged biomolecular species associated with the block copolymer. The block copolymer can have any composition, structure, and/or properties described in Section I above. The negatively charged biomolecular can be associated with a plurality of individual block copolymer chains, in some embodiments. The number of block copolymer chains associated with the negatively charged biomolecular species can be dependent on several considerations including specific identity of the charged biomolecular species, and specific structure and cationic charge of the block copolymer. Additionally, a polyion complex can exhibit a N/P ratio of 1 to 20, in some embodiments.

The negatively charged biomolecular species, in some embodiments, comprises one or more nucleic acids and/or oligonucleotides, including DNA and RNA. Non-limiting species of DNA can comprise single-stranded DNA (ss-DNA), double-stranded DNA (ds-DNA), and plasmid DNA (p-DNA). Non-limiting species of RNA can comprise messenger RNA (mRNA), transfer RNA (RNA), ribosomal RNA (rRNA), small interfering RNA (siRNA), circular RNA (circRNA), and small-activating RNA (sa-RNA). The nucleic acids and/or oligonucleotides can be synthetic or natural. Nucleic acids, in some embodiments, comprise one or more modifications. For example, synthetic mRNA can comprise one or more uridine analogs, such as pseudouridine (Ψ) and/or N¹-methyl-pseudourine (m1ψ). Additionally, modifications of nucleic acids and/or oligonucleotides described herein also include protein modifications. In some embodiments, a nucleic acid or oligonucleotide is modified with one or more proteins, including enzymes such as nucleases. For example, a nucleic acid of a polyion complex described herein can be Cas9 gRNA.

The polyion complex can exhibit particle-like morphology having a hydrodynamic diameter of 50 nm to 150 nm. In some embodiments, polyion complexes described herein can have a hydrodynamic diameter of 70 nm to 120 nm.

### III. Dispersions

In another aspect, dispersions are described herein. In some embodiments, a dispersion comprises an aqueous or aqueous-based continuous phase, and a dispersed phase comprising polyion complexes, the polyion complexes comprising a block copolymer including a hydrophilic block comprising oxazoline monomer or oxazine monomer, and a cationic block comprising monomer including a linear or branched cationic polyamine side chain, and a negatively charged biomolecular species associated with the block copolymer. The block copolymer can have any composition, structure, and/or properties described in Section I above. Moreover, the negatively charged biomolecular species can have any composition and/or properties described in Section II above. The negatively charged biomolecular species, for example, can comprise one or more nucleic acids and/or oligonucleotides. The negatively charged biomolecular species, in some embodiments, can be present in the dispersion in at a concentration ranging from 0.01 to 10 mg/mL. In some embodiments, the negatively charged biomolecular species can be present in the dispersion in at a concentration having a value selected from Table I.

```
TABLE I
Biomolecular Species Concentration (mg/mL)
0.1-5
0.5-5
  1-10
0.05-0.4
0.1-0.3
0.15-0.3
0.2-0.3
  0.1-0.25
```

As provided in Section II, the polyion complexes of the dispersion can exhibit an average hydrodynamic diameter of 10 nm to 300 nm. In some embodiments, the polyion complexes have an average hydrodynamic diameter selected from Table II.

TABLE II
Polyion Complex Average Hydrodynamic diameter
30-250 nm
50-200 nm
70-150 nm
80-120 nm
90-110 nm
95-105 nm

In addition to the foregoing average hydrodynamic diameter, the polyion complexes of the dispersion can exhibit a PDI of 0.1-0.3 or 0.1-0.2. In some embodiments, PDI of the polyion complexes in the dispersion can be less than 0.1, such as 0.01-0.09.

Dispersions described herein, in some embodiments, further comprise an amphiphilic excipient. Any amphiphilic excipient consistent with the technical objectives described herein can be employed. In some embodiments, the amphiphilic excipient comprises one or more polar lipids. Suitable polar lipids can be natural or synthetic, branched and/or comprise one or more points of unsaturation. Polar lipids of the dispersion can also be negatively charged, in some embodiments. In some embodiments, suitable polar lipids include cardiolipin species such as 16:1 cardiolipin or 18:1 cardiolipin. Polar lipids can be operable to interact with polyion complexes described herein. In some embodiments, presence of the third hydrophobic block can facilitate interaction with the polar lipids. The amphiphilic excipient can be present in the dispersion in any desired amount. In some embodiments, the amphiphilic excipient, including one or more polar lipids, is present in the dispersion in an amount of 0.5% w/w to 10% w/w.

Dispersions described herein can also comprise one or more counterion species to the cationic polyamine side chains of the block copolymer. When present, the counterions can interact with the cationic polyamine side chains, thereby shielding excess positive charges of the block copolymer under conditions of high N/P ratios. Such shielding can stabilize the polyion complexes resulting in more compact complexes with lower PDI. Any negatively charged counterion consistent with the technical objectives described herein can be employed. In some embodiments, the counterion comprises sulphate (SO₄⁻²), sulfonic acid moiety (—SO₃⁻) or combinations thereof. Counterion species can be provided as salts to the dispersion, such as alkali metal salts and/or alkaline earth metal salts. Counterion species may also be provided as buffer added to the dispersion. In some embodiments, counterion is present in the dispersion at a concentration of 0.5 mM to 10 mM.

Dispersions described herein can also comprise buffer. Any buffer consistent with the technical objectives described herein can be employed. In some embodiments, identity of the buffer is chosen on the ability of the anion to interact with the cationic polyamine side chains, as provided above. Suitable buffer can be selected from PBS, HEPES and TBE, in some embodiments. Additionally, in some embodiments, buffer concentration can be 0.5 mM to 10 mM. Presence of buffer can significantly enhance stability of the polyion complexes. Polyion complexes of dispersions comprising buffer, in some embodiments, can maintain substantially the same average hydrodynamic diameter and/or PDI for a period of at least 72 hours. In being substantially the same, the average particle hydrodynamic diameter and PDI of the polyion complexes of buffered dispersions are within 100 nm and 0.05-0.1 of the polyion complexes after initial formation.

Dispersions described herein, in some embodiments, are isotonic for in vivo applications. In such embodiments, the dispersions can include one or more saccharides. The one or more saccharides can be a substitute for sodium chloride in the isotonic composition. In some embodiments, glucose is employed in the dispersion. One or more saccharides can be present in addition to one or more of the counterion species and buffer.

As described further herein, the dispersion can be lyophilized, stored, and reconstituted at a later date. When reconstituted via hydration, the dispersion exhibits average polyion complex hydrodynamic diameter and PDI having substantially the same values as pre-lyophilization. In being substantially similar, the average particle hydrodynamic diameter and PDI of the polyion complexes of the reconstituted dispersion are within 10-50 nm and 0.05-0.1 of the polyion complexes prior to lyophilization. Additionally, lyophilization does not affect transfection capabilities of the polyion complexes described herein.

### IV. Methods of Producing Dispersions

In another aspect, methods of producing dispersions are described herein. A method of forming a dispersion, in some embodiments, comprises providing a dried film comprising a block copolymer, the block copolymer including a hydrophilic block comprising oxazoline monomer or oxazine monomer, and a cationic block comprising monomer including a linear or branched polyamine side chain. The cationic block copolymer can have any composition, structure, and/or properties described in Sections I-III above.

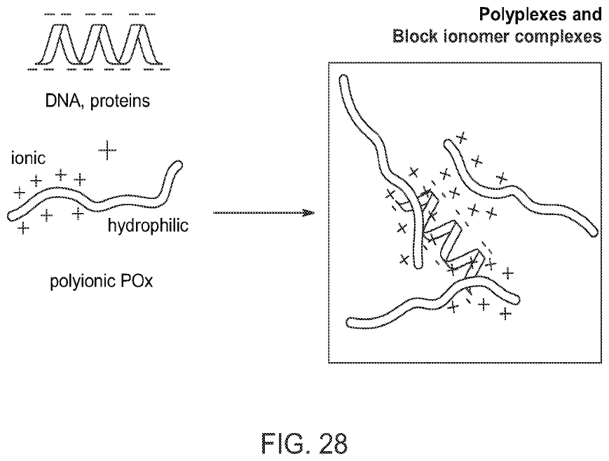

The dried film is contacted with an aqueous or aqueous-based continuous phase comprising a negatively charged biomolecular species, thereby forming a dispersed phase of polyion complexes in the continuous phase, the polyion complexes comprising the negatively charged biomolecular species associated with the block copolymer. Gentle agitation may be employed after the dried film is contacted with the continuous phase. FIG. 28 illustrates a method of forming a dispersion according to some embodiments described herein.

The polyion complexes can have any composition, structure, and/or properties described in Sections II-III above. In some embodiments, the negatively charged biomolecular species comprises a nucleic acid, such as DNA or RNA, as well as oligonucleotides. Additionally, in some embodiments, the dried film further comprises an amphiphilic excipient. The amphiphilic excipient becomes part of the dispersion following contact with the aqueous or aqueous-based continuous phase. In some embodiments, the amphiphilic excipient comprises one or more polar lipids, as described in Section III above. Moreover, counterion and/or buffer can be added to the continuous phase prior to contacting the dried film. The dispersion, in some embodiments, is lyophilized, stored, and reconstituted at a later date, as described in Section III above.

These and other embodiments are further illustrated in the following non-limiting examples. In the following Examples and as described herein, polyion complexes and polyplexes are interchangeable.

### Example 1—Block Copolymers

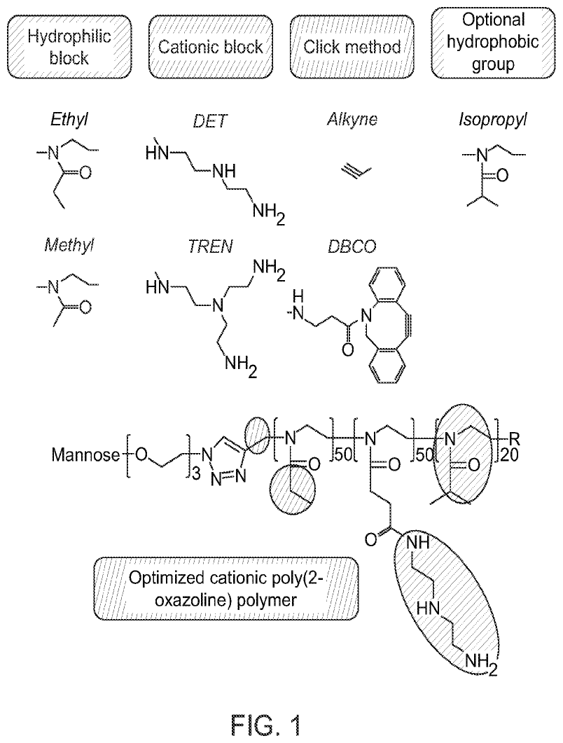

To design a PEG-free polymer for plasmid transfection of immune cells, such as macrophages, several poly(2-oxazoline)-based cationic copolymers were developed by varying non-ionic hydrophilic, cationic, and hydrophobic blocks, and employing azide-alkyne cycloaddition (“click chemistry”) methods for the attachment of the targeting moiety (FIG. 1). The polymers were synthesized by sequential living cationic ring-opening polymerization (LCROP) of 2-oxazoline monomers which provides access to a wide range of polymer structures with defined molecular mass and narrow dispersity (1.01-1.30). (See R. Luxenhofer, A. Schulz, C. Roques, S. Li, T. K. Bronich, E. V. Batrakova, R. Jordan, A. V. Kabanov, Biomaterials 2010, 31, 4972.)

Copolymers were synthesized in acetonitrile (ACN) via sequential LCROP carried out in optimal glovebox conditions with H₂O and O₂ levels always maintained below 10 ppm and 20 ppm, respectively. Monomers were purchased from Sigma-Aldrich (St. Louis, MO). Polymers were synthesized in ACN with the initiators propargyl p-toluenesulfonate or p-toluenesulfonic acid methyl ester using the following purified monomers: 2-ethyl-2-oxazoline (EtOx), 2-methyl-2-oxazoline (MeOx), 2-methoxy-carboxyethyl-2-oxazoline (MestOx), and 2-isopropyl-2-oxazoline (iPrOx). Cationic modifications were made with diethylenetriamine (DET) or tris(2-aminoethyl) amine (TREN). Polymers were terminated with either 3-Amino-1-[(5-aza-3,4:7,8-dibenzocyclooct-1-yne)-5-yl]-1-propanone (dibenzocyclooctyne-amine or DBCO-amine) or piperidine. Alpha-Mann-TEG-N3 (Iris Biotech, Marktredwitz, Germany) (mannose) was conjugated as a targeting moiety to the alkyne via click chemistry.

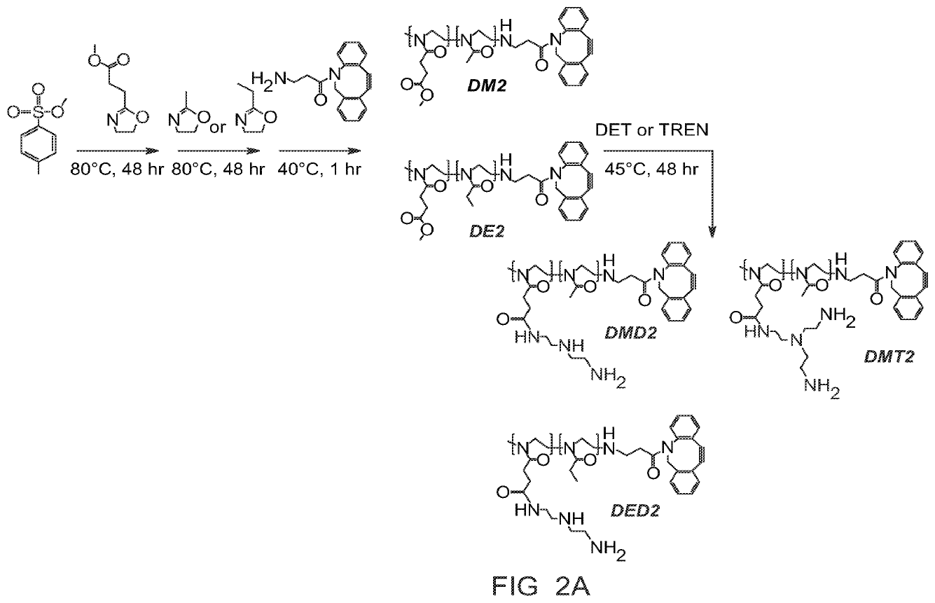

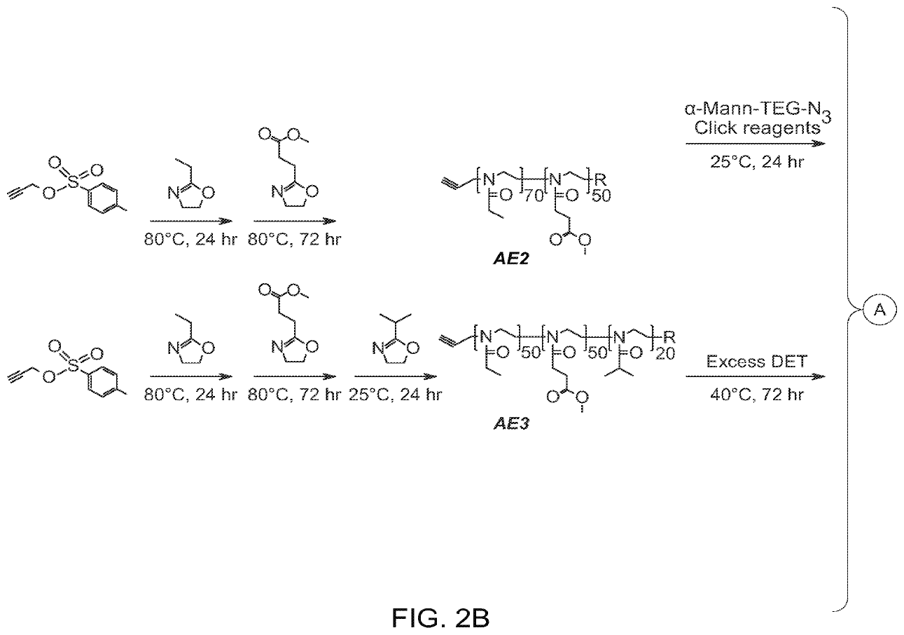

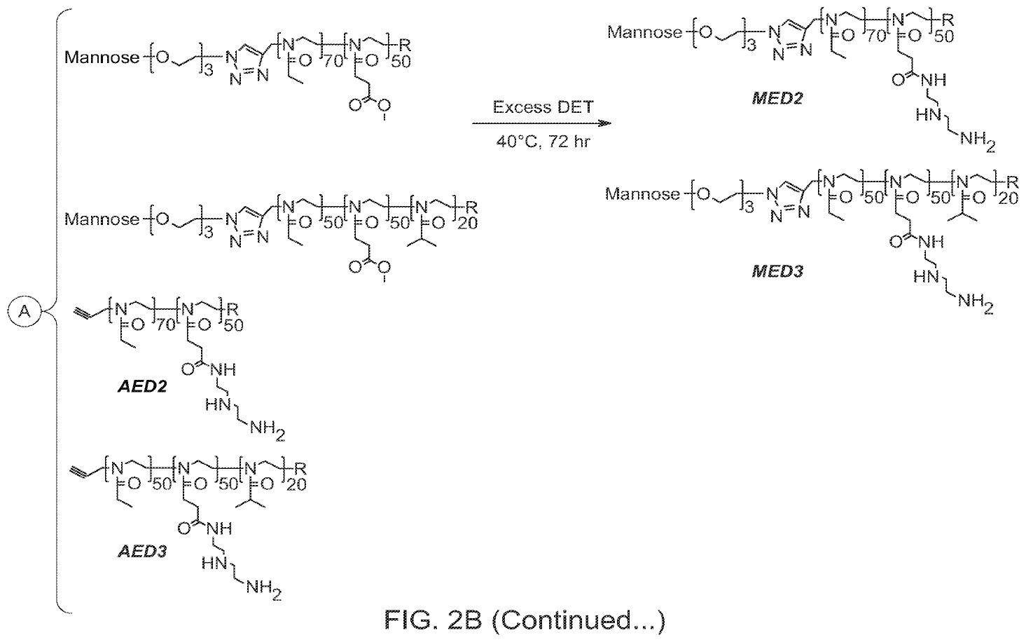

Two different strategies to introduce “clickable” groups were used for targeting moieties attachment to the free ends of the hydrophilic blocks. In one strategy, shown in FIG. 2A, p-toluenesulfonic acid methyl ester was employed as the initiator, and the cationic block precursor pMestOx was first polymerized, followed by the hydrophilic block, which is terminated by DBCO-amine for copper-free click chemistry. In the second strategy, shown in FIG. 2B, alkyne-containing propargyl p-toluenesulfonate was employed as the initiator and then the hydrophilic block and the cationic block precursor that was terminated by piperidine was sequentially polymerized. The hydrophilic block structure was varied using either MeOx or FtOx as the monomers (FIG. 2A). A third type of relatively hydrophobic block was also introduced by polymerizing iPrOx after the cationic precursor (FIG. 2B), to enhance block copolymer self-assembly during polyplex formation. After synthesis of the block copolymer precursor, the mannose targeting moiety Alpha-Mann-TEG-N3 was attached using copper-free (FIG. 2A) or copper-catalyzed (FIG. 2B) click chemistry. Finally, the cationic moieties were introduced by reacting the methyl ester groups of the corresponding block copolymer precursors with either DET or TREN.

The DBCO-containing block copolymers (FIG. 2A), DBCO-pMeOx₇₀-pMestOx(DET)₅₀ (DMD2), DBCO-pEtOx₇₀-pMestOx(DET) 50 (DED2), and DBCO-pEtOx₇₀-pMestOx(TREN) 50 (DMT2), were synthesized as follows. The reaction was initiated by p-toluenesulfonic acid methyl ester (0.238 mmol, 1 eq.) followed by sequential polymerization of MestOx (11.89 mmol, 50 eq.) and either MeOx (16.67 mmol, 70 eq.) or EtOx (16.67 mmol, 70 eq.). Monomers were sequentially added to the reaction mixture dropwise and stirred at 80° C. for two days for the first and second blocks. The reaction was terminated by DBCO-amine (0.714 mmol, 3 eq.). The resulting polymer precursors DMD2 (MeOx block) or DED2 (EtOx block) were modified with DET or TREN by stirring each mannosylated polymer (20 mg) in a DET or TREN solution (2 mL) at 40° C. for three days. Excess DET was purified by dialysis and final polymers DMD2 and DED2, modified by DET, and DMT2, modified by TREN, were collected by lyophilization, and stored at −20° C.

The alkyne-containing block copolymers (FIG. 2B), pEtOx₇₀-pMestOx(DET) 50 (AED2) and Alkyne-pEtOx₅₀-pMestOx(DET) 50-piPrOx₂₀ (AED3), were synthesized as follows. The reaction was initiated by propargyl p-toluenesulfonate (0.238 mmol, 1 eq.) in acetonitrile (5 mL) followed by sequential polymerization of EtOx (AED2: 16.67 mmol, 70 eq., AED3: 11.89 mmol, 50 eq.,) and then MestOx (11.89 mmol, 50 eq.), and, in case of triblock copolymer, iPrOx (4.76 mmol, 20 eq,). All monomers were added to the reaction media dropwise and the reaction was carried upon constant stirring at 80° C. overnight for the first block, three days for the second block, and overnight for the third block. The reactions were terminated by piperidine (0.714 mmol, 3 eq.) which was added after completion of either second or third block. After each polymerization step and reaction termination, the precursor polymers (diblock, AE2, or triblock, AE3) were characterized with 1H NMR. Acetonitrile was removed in vacuo. Finally, precursors AE2 or AE3 were either conjugated to mannose and then modified with DET, or just modified with DET. For non-mannosylated polymers, excess DET was added to precursors AE2 and AE3 by stirring dry polymer (20 mg) in a DET solution (2 mL) at 40° C. for 3 days. Excess DET was purified by dialysis against 3.5 kDa MWCO membrane in 0.01N HCl overnight followed by dialysis in DI water for 2 days. Diblock AED2 and triblock AED3 were lyophilized and stored at −20° C.

The mannose-pEtOx₇₀-pMestOx(DET) 50 (MED2) and mannose-pEtOx₅₀-pMestOx(DET) 50-piPrOx₂₀ (MED3) were prepared as follows (FIG. 2B). Alpha-Man-TEG-N3 (mannose) was conjugated to the ethyl oxazoline block via CuAAC click chemistry. Stock mannose was diluted with DI water to 200 mg/mL and stock solutions were kept at −20° C. After reconstituting AE2 or AE3 (50 mg) in DI water, mannose (16.9 mg) was added, and the solution was stirred for 5 min. Next, CuSO₄*5H₂O (2.4 mg) and sodium ascorbate (3.0 mg) were added sequentially. Volume of final solutions was kept at 1 mL and stirred at room temperature (RT) overnight. Next, solutions were dialyzed against DI water in a 3.5k MWCO membrane for 2 days. MED2 and MED3 were then lyophilized and analyzed using NMR. Mannose ligand conjugation yield: 37% for MED2, 31% for MED3; 1H NMR (400 MHz, D₂O, 25° C.).

The resulting polymers are presented in Table 1.

TABLE 1
Synthesized Polymers

| | | | | | | |
|---|---|---|---|---|---|---|
| Abbreviation | Polymer Structure | (x) | (y) | (z) | Mₙ (kDa) (NMR) | Precursor |

Block copolymer precursors

| | | | | | | |
|---|---|---|---|---|---|---|
| DM2 | DBCO-P(MeOx)ₓ-b-(MestOx)_{y} | 70 | 50 | — | 13.3 | — |
| DE2 | DBCO-P(EtOx)ₓ-b-(MestOx)_{y} | 70 | 50 | — | 14.3 | — |
| AE2 | Alkyne-P(EtOx)ₓ-b-(MestOx)_{y} | 70 | 50 | — | 14.1 | — |
| AE3 | Alkyne-P(EtOx)ₓ-b-(MestOx)_{y}-b-(iPrOx)_{z} | 50 | 50 | 20 | 14.4 | — |
| M1 | Methyl-P(EtOx)ₓ-b-(Mest-DET)_{y} | 70 | 50 | | 19.5 (calculated) | — |
| M2 | Methyl-P(EtOx)ₓ-b-(BuOx)_{y}-b-(Mest-DET)_{z} | 35 | 20 | 30 | 13.5 (calculated) | — |
| M3 | Methyl-P(EtOx)ₓ-b-(BuOx)_{y}-b-(Mest-DET)_{z} | 35 | 10 | 30 | 12.0 (calculated) | — |

Cationic block copolymers

| | | | | | | |
|---|---|---|---|---|---|---|
| DMD2 | DBCO-P(MeOx)ₓ-b-(MestOx(DET))_{y} | 70 | 50 | — | 17.7 | DM2 |
| DMT2 | DBCO-P(MeOx)ₓ-b-(MestOx(TREN))_{y} | 70 | 50 | — | 19.8 | DM2 |
| DED2 | DBCO-P(EtOx)ₓ-b-(MestOx(DET))_{y} | 70 | 50 | — | 18.6 | DE2 |
| AED2 | Alkyne-P(EtOx)ₓ-b-(MestOx(DET))_{y} | 70 | 50 | — | 18.5 | E2 |
| AED3 | Alkyne-P(EtOx)ₓ-b-(MestOx(DET))_{y}-b-(iPrOx)_{z} | 50 | 50 | 20 | 18.0 | E3 |
| MED2 | Mannose-P(EtOx)ₓ-b-(MestOx(DET))_{y} | 70 | 50 | — | 18.8 | E2 |
| MED3 | Mannose-P(EtOx)ₓ-b-(MestOx(DET))_{y}-b-(iPrOx)_{z} | 50 | 50 | 20 | 18.3 | E3 |
| D | Methyl-P(EtOx)ₓ-b-(Mest-DET)_{y} | 70 | 50 | — | 23.0 (calculated) | M1 |
| TL | Methyl-P(EtOx)ₓ-b-(BuOx)_{y}-b-(Mest-DET)_{z} | 35 | 20 | 30 | 15.6 (calculated) | M2 |
| TS | Methyl-P(EtOx)ₓ-b-(BuOx)_{y}-b-(Mest-DET)_{z} | 35 | 10 | 30 | 14.2 (calculated) | M3 |

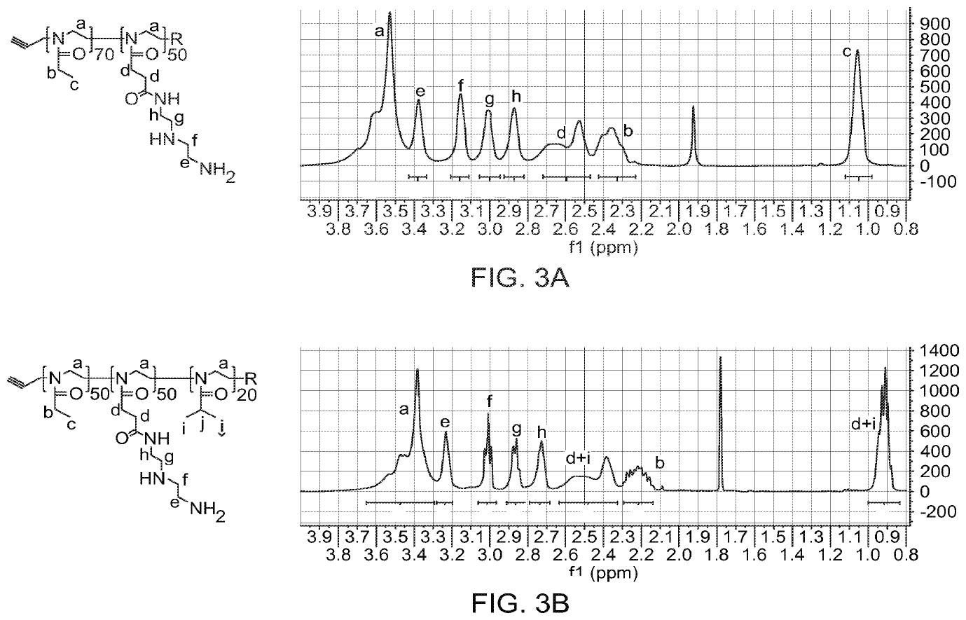

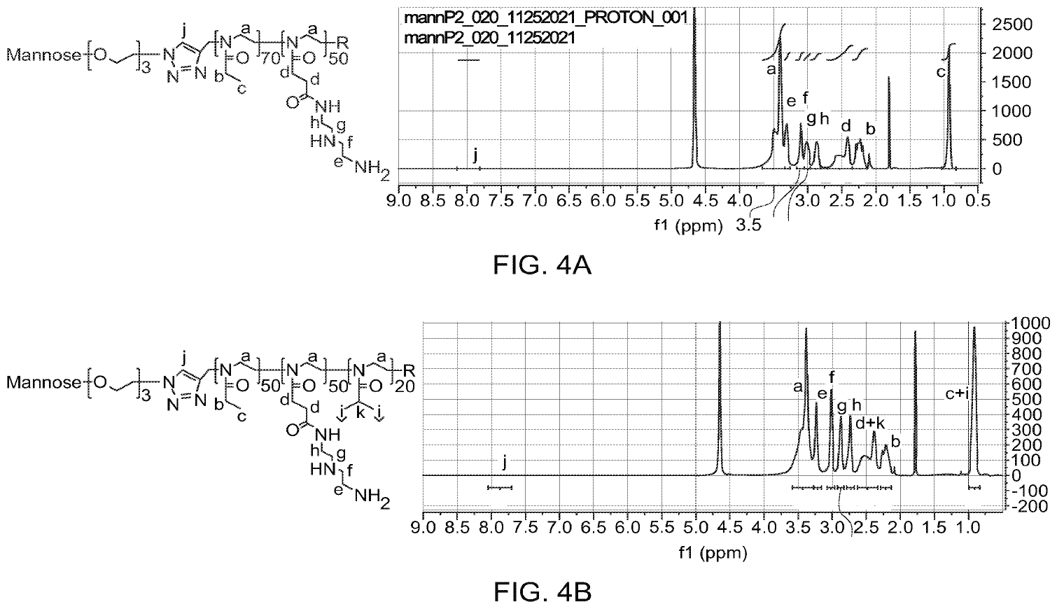

Mannose conjugation was confirmed via NMR (FIG. 3A, FIG. 3B, FIG. 4A, and FIG. 4B). ¹H-NMR spectra were recorded on an INOVA 400 (Agilent Technologies, Santa Clara, CA) at room temperature. The spectra were calibrated using the solvent signals (D₂O 4.80 ppm). 1H-NMR was used to calculate molecular number weight of the polymer and polymer precursors.

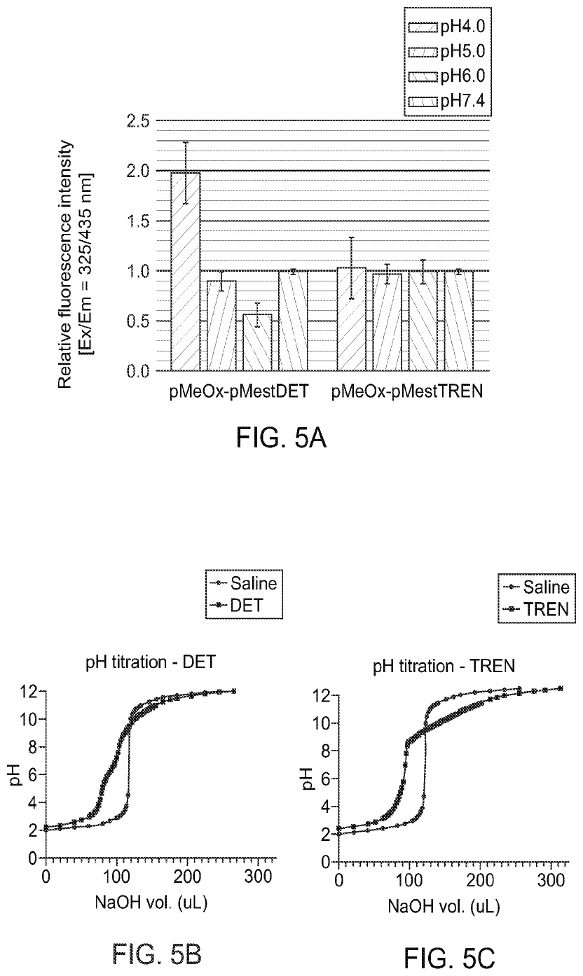

Polymers were characterized with 2-(p-toluidino)-6-naphthalene sulfonic acid (TNS) assay, pH titration, and by examining buffering capacity (FIG. 5A, FIG. 5B, and FIG. 5C). The TNS was from Millipore (Sigma). The TNS assay was further used to examine the DET and TREN-containing MeOx block copolymers. TNS fluorescence increases upon binding to the protonated amines. Polyplexes were prepared in 10 mM citrate buffer (300 μL), containing 150 mM NaCl, at pH 4.0, 5.0, 6.0, and 7.4 and mixed with 3 μL of 6 mM TNS using vortex mixing. Fluorescence intensity of samples was measured in triplicate with 100 μL volume per well in a 96-well black plate at Ex/Em 325/435 nm using a SpectraMax M5 plate reader (Molecular Devices, San Jose, CA). Fluorescence intensity was normalized to fluorescence at pH 7.4. The fluorescence intensity of TNS upon mixing with the TREN-containing copolymer was constant across the pH 4.0 to pH 7.4 range, which suggests that the TREN side chains were protonated in this range pH. In contrast for the DET-containing copolymer, the fluorescence signal increased at lower upon acidification from pH 5.0 to pH 4.0, with is indicative of the amino group protonation in this range (FIG. 5A).

For the acid-base titration assay, cationic block copolymers were dissolved in 10 mM HCl-containing saline at the cationic repeating unit base-molar concentration of 3 mM (the base-molar concentration represents the polymer molar concentration multiplied by the degree of polymerization of the cationic block). Initial pH 2 was recorded and small amounts of 0.1 M NaOH were added while measuring pH after each addition until reaching pH 12. To analyze the buffering capacity, the change in dOH⁻ was divided by dpHI for each measurement in the titration. The resulting value indicates how much OH is needed to increase pH. The pH titration study suggests that these polymers display buffering capacity in a broad range of pH indicative of protonation of multiple amino groups. Specifically, the methyl-based DET-containing copolymer displays buffering capacity in both acidic and alkali areas with effective pKa values of approximately 6.0 and 11.0, while the TREN-containing polymer displayed a buffering capacity in the ranges corresponding to effective pKa of approximately 4.0 and 10.0. (FIG. 5B and FIG. 5C). Ethyl-based DET-containing copolymer also displayed a buffering capacity in both acidic and alkali regions with effective pKa close to 4.3 to 8.8 respectively.

### Example 2—Polyion Complexes (Polyplexes)

To produce the polyion complexes, the cationic copolymers were mixed with luciferase-encoding pDNA using simple vortex mixing at various N/P ratios and incubated at room temperature for 30 minutes prior to any characterization. Polyplexes were formed using polymers and gWIZ™ luciferase-encoding plasmid (luc-pDNA) (Gene Therapy Systems, San Diego, CA) and expanded using Plasmid Giga Kit (Qiagen, Hilden, Germany) following the supplier's protocol. To obtain different N/P ratios the amount of luc-pDNA was kept constant (33 ug/mL) while the concentration of the polymer was varied in each polyplex formulation. Polymers were serially diluted with 10 mM HEPES buffer according to desired N/P ratio and briefly mixed with the fixed amount of luc-pDNA using vortex mixer. Polyplexes were incubated at RT for 30 min. For further characterization at physiological conditions, 3M NaCl was added to polyplex solutions to reach final concentration of 150 mM NaCl. Those solutions were then incubated at 37° C. for 60 min.

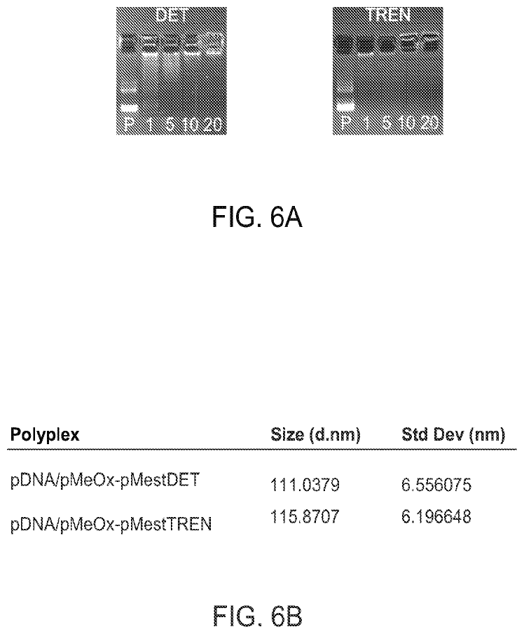

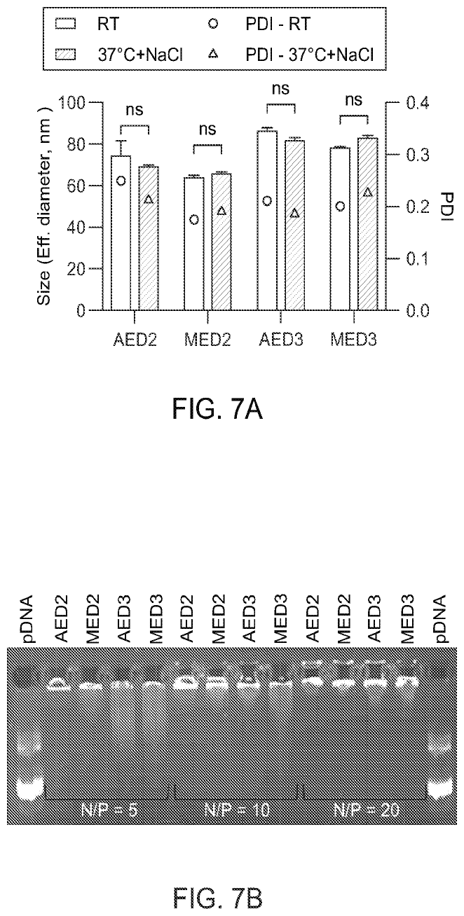

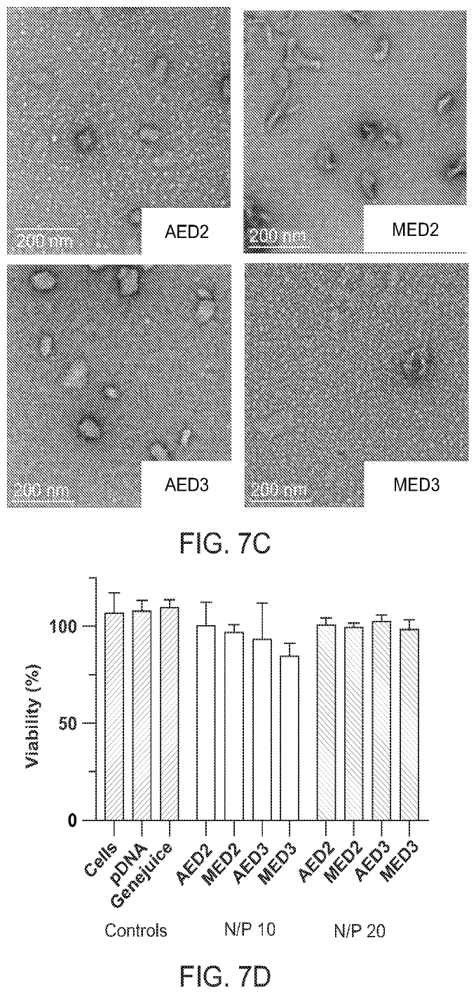

The formation of the polyplexes was detected by the changes of the electrophoretic mobility of the luc-pDNA in 1% agarose gel, by particle size measurements using dynamic light scattering (DLS) as well as TEM (FIG. 6A, FIG. 7A, FIG. 7B, and FIG. 7C). To confirm luc-pDNA complexation with polymers, gel electrophoresis was performed (FIG. 6A and FIG. 7B). Polyplexes were mixed with 6× orange loading dye (Thermo Fisher Scientific, Waltham, MA) prior to running agarose gel electrophoresis in 1×TAE buffer to confirm complexation. Samples were loaded onto 1% agarose gel in 1×TAE buffer and run at 100V for 45 min. Experimental samples were compared to naked luc-pDNA alone. Ethidium Bromide (EtBr) (Thermo Fisher Scientific, Waltham, MA) was added to the agarose gel for visualizing the luc-pDNA under UV illumination.

The Z-average hydrodynamic diameter and polydispersity index (PDI) were determined by dynamic light scattering (DLS) using a Malvern Zetasizer (Malvern Instruments, Westborough, MA). Samples for DLS were prepared in 10 mM HEPES buffer (50 μL) and measured in triplicate with a minimum of 10 runs per measurement per sample. Measurements were taken either after 30 min incubation at RT or after 30 min incubation at RT followed by 60 min incubation at 37° C. Generally, the particle sizes for polyplexes of various compositions varied from ca. 70 to ca. 120 nm with fairly narrow polydispersity index (PDI ca. 0.2) (FIGS. 6B and 7A).

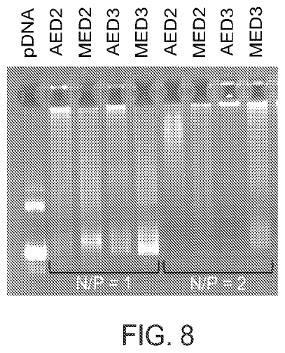

To examine the morphology, polyplexes were prepared at N/P 20 and then imaged with TEM. All TEM images were obtained on a Talos F200X S/TEM microscope (Thermo Fisher Scientific, Waltham, MA). Polyplex samples prepared at N/P 20 were applied to 300 mesh carbon-coated copper grids and stained with 4% uranyl acetate prior to imaging (Ted Pella, Redding, CA). Excess sample was blotted gently and allowed to air dry prior to imaging. The complexes were distinct, non-aggregated and either spherical or somewhat elongated (short worms) (FIG. 7C). No difference in size was observed between polymers with MeOx block compared to EtOx block (FIG. 6B). Thus, at lower N/P ratios the DET containing diblock copolymers displayed some disproportioning—i.e., presence of free luc-pDNA or negatively charged complexes that were mobile in gels, along with the polyplexes remaining at the start of the gel (FIG. 8). The TREN containing diblock copolymers revealed greater propensity for formation of the complexes than the DET containing copolymers. This was likely due to higher charge density of the TREN (three chargeable amino groups) vs DET (two chargeable amino groups). Addition of the third hydrophobic piPrOx block in the copolymer increased the tendency for disproportioning. The triblock copolymer AED3 (P(EtOx)₅₀-b-(MestOx(DET))₅₀-b-(iPrOx)₂₀-Alkyne) did not form complexes well at lower N/P ratios of 1 and 2, although the complexation at N/P 10 and 20 was nearly complete (FIG. 7B and FIG. 8). Another factor impacting copolymer binding to the luc-pDNA was attachment of the mannose. At lower N/P ratios of 1 and 2 this appeared to hinder the polyion complexation of mannosylated block copolymers with pDNA in contrast to the non-mannosylated counterparts (FIG. 8). The least effective binding was observed with the mannosylated triblock MED3 (P(EtOx)₅₀-b-(MestOx(DET))₅₀-b-(iPrOx) 20-Mannose). For this copolymer, some mobility of the luc-pDNA in the gel was seen even at highest N/P ratios 10 and 20 (FIG. 7B).

For zeta-potential evaluation, polyplexes were prepared at N/P 20 and incubated at RT for 30 min. Samples were diluted with DI water (total volume 1 mL) and measured on a Malvern Zetasizer (Malvern Instruments, Westborough, MA). With all copolymers at such high N/P ratios the polyplexes were positively charged according to the zeta-potential measurements.

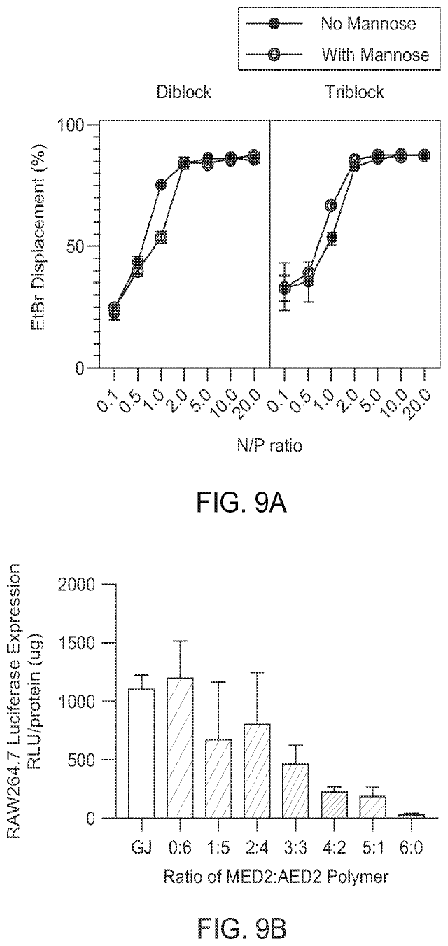

To understand the complexation of mannosylated polymers further, polyplexes were tested by an ethidium bromide (EtBr) displacement assay. By forming polyplexes with a mixture of EtBr and luc-pDNA, the polymer competes against EtBr which allows us to monitor luc-pDNA condensation. Briefly, EtBr was diluted to 2 μg/mL and mixed with luc-pDNA to a final luc-pDNA concentration of 33 μg/mL. Polyplexes were formed in an opaque 96-well black plate at various N/P ratios ranging from 0.1 to 20. EtBr displacement was quantified by measuring fluorescence at Ex/Em 520/590 nm emission using a SpectraMax M5 plate reader (Molecular Devices, San Jose, CA). Relative fluorescence (%) was calculated by subtracting EtBr alone background fluorescence from each experimental sample and normalizing to fluorescence of a control solution containing only luc-pDNA and EtBr. Though gel electrophoresis showed a lack of complexation at lower N/P ratios of 1 and 2 for polyplexes based on both diblock and triblock copolymers, EtBr displacement revealed that these same polyplexes displaced EtBr successfully starting at N/P ratio 2 (FIG. 9A). Both diblock-based and triblock-based polyplexes showed similar EtBr displacement levels at ˜85% displacement. Interestingly, polymer mannosylation did not affect the amount of EtBr displaced.

### Example 3—Polyion Complex Transfection of Macrophages

All polyplexes were screened in vitro for transfection efficiency. Transfection assays allowed the comparison of the cationic blocks between polymers DMD2 (pMeOx₇₀-pMestOx(DET)₅₀-DBCO) or DMT2 (pMeOx₇₀-pMestOx(TREN)₅₀-DBCO), which have either DET or TREN cationic moieties. IC21 macrophages were cultured in RPMI media supplemented with 10% FBS and 1% p/s. All cell cultures were maintained at 37° C. and 5% CO₂. Polyplex formulations were prepared at N/P ratios 10 and 20. IC21 cells were seeded in a 24-well plate. After reaching 70% confluency, serum-containing media was replaced with serum-free DMEM. Cells were treated with luc-pDNA alone, GeneJuice (EMB Millipore Novagen, Madison, WI, as positive control), or polyplexes for 24 hours with each well receiving a total of luc-pDNA (1 ug). After treatment, cells were rinsed once in DPBS (500 μL) and lysed in 1× cell culture lysis buffer (100 μL) for 45 min on a shaker plate at room temperature. Lysates were collected and either immediately analyzed for luciferase activity or stored at −80° C. for further analysis.

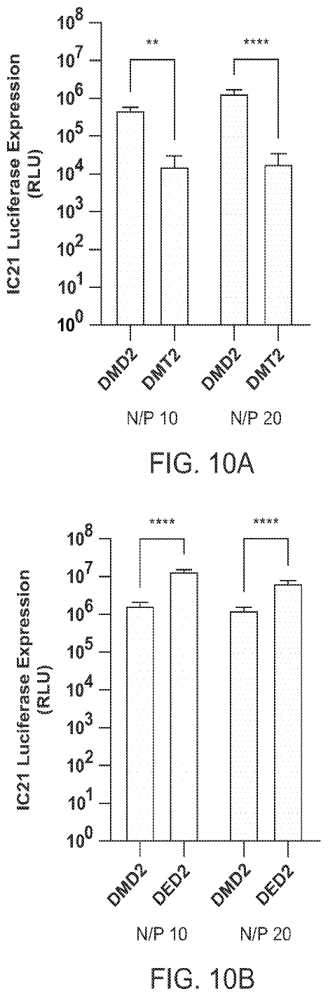

After transfection, cell lysates were analyzed for luciferase activity with dual-assay reporter kit (Promega, Madison, WI) following the manufacturer protocol. All samples were measured in triplicate. Final luciferase activity was reported as RLU. The DMD2 based polyplexes transfected cells significantly better than DMT2 based polyplexes at both N/P 10 (p<0.01) and N/P 20 (p<0.0001) (FIG. 10A). Therefore, DET was selected over TREN as a cationic moiety in the polycation in further experiments. Copolymers differing in hydrophilic MeOx or EtOx block, DMD2 (pMeOx₇₀-pMestOx(DET)₅₀-DBCO) and DED2 (pEtOx₇₀-pMestOx(DET)₅₀-DBCO), were also compared in in vitro transfection. DED2 was significantly better at transfecting IC21 cells than DMD2 at both N/P 10 (p<0.0001) and N/P 20 (p<0.0001) (FIG. 10B). These head-to-head comparisons prompted the choice of the cationic block DET and the hydrophilic block EtOx for subsequent polymer design.

The transfection efficiency of polyplexes made from AED2, MED2, AED3, and MED3 were subsequently tested in RAW264.7 macrophages and bone marrow derived macrophages (BMDM). RAW264.7 macrophages were cultured in DMEM media supplemented with 10% FBS and 1% penicillin/streptomycin (p/s). Bone marrow-derived macrophages (BMDM) (129/sv background) were isolated from the femur of a mouse. The monocytes were cultured for 10 days in DMEM media supplemented with 10% FBS and MCSF-containing media obtained from L929 cells. BMDMs were used on Day 10. All cell cultures were maintained at 37° C. and 5% CO₂. Polyplex formulations were prepared at N/P ratios 10 and 20. RAW264.7 or primary BMDM cells were seeded in a 24-well plate. After reaching 70% confluency, serum-containing media was replaced with serum-free DMEM. For cytotoxicity analysis, RAW264.7 cells were treated with media, luc-pDNA alone, GeneJuice, or polyplexes for 24 hours with each well containing 0.25 μg luc-pDNA. After incubation, fresh serum-containing DMEM was applied containing 10% CCK-8 solution. Cytotoxicity was evaluated with a CCK-8 assay (Dojindo, Rockville, MD). Absorbance was read at 450 nm after 1 hr. The copolymer-based polyplexes were non-toxic to cells even at higher concentrations used (high N/P ratios for polyplexes) (FIG. 7D).

For transfection efficiency analysis, cells were treated with luc-pDNA alone, GeneJuice (as positive control), or polyplexes for 24 hours with each well receiving a total of luc-pDNA (1 μg). After treatment, cells were rinsed once in DPBS (500 μL) and lysed in 1× cell culture lysis buffer (100 μL) for 45 min on a shaker plate at room temperature. Lysates were collected and either immediately analyzed for luciferase activity or stored at −80° C. for further analysis. After transfection, cell lysates were analyzed for luciferase activity with dual-assay reporter kit (Promega, Madison, WI) following the manufacturer protocol. To normalize luciferase activity results per well, total protein was quantified using the Pierce™ BCA assay kit (Thermo Fisher Scientific, Waltham, MA) in a 96-well plate. Final luciferase activity was normalized by total protein in cell sample and reported as RLU/ug total protein.

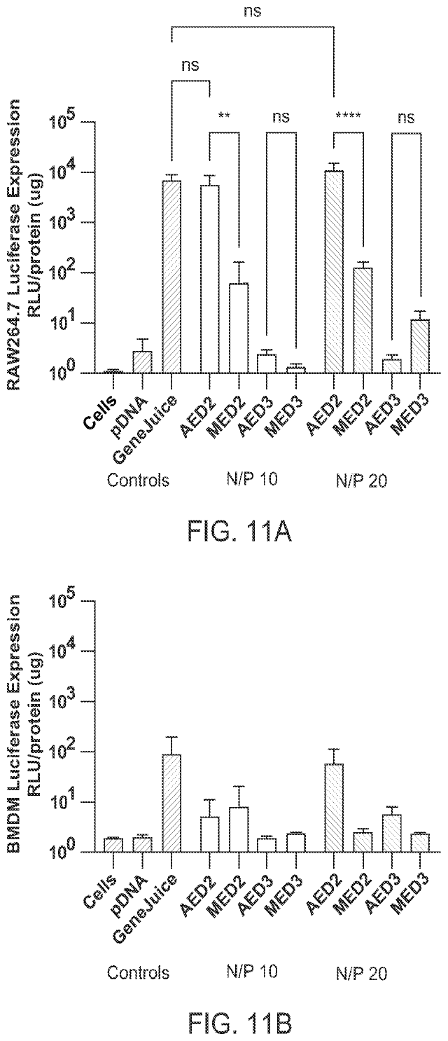

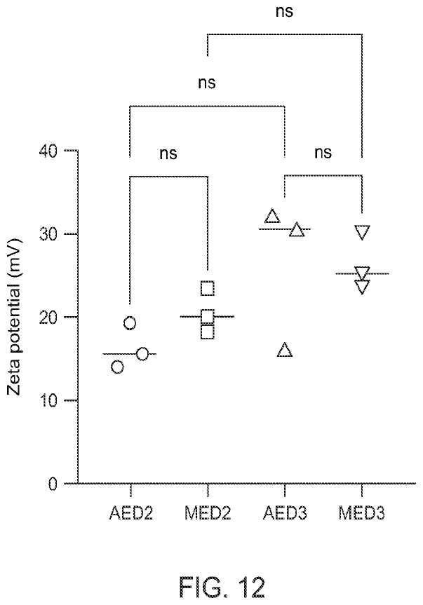

The AED2-based polyplexes outperformed the polyplexes made using other polymers in both RAW264.7 and BMDM transfection at both N/P ratios 10 and 20 (FIG. 11A). Surprisingly, the mannosylated MED2-based polyplexes performed significantly worse than its non-mannosylated AED2-based counterparts at both N/P 10 (p<0.01) and N/P 20 (p<0.0001). Polyplexes made with AED2 at N/P 10 and 20 transfected RAW264.7 macrophages similarly to the commercial transfection reagent GeneJuice (n.s). A similar trend was seen in BMDMs; AED2-based polyplexes at N/P 20 performed comparably to the positive control, GeneJuice, although the overall levels of luciferase reporter gene expression normalized to the total protein were much less than those in RAW264.7 macrophages (FIG. 11B). Neither of the mannose-free or mannosylated triblock copolymers, AED3 and MED3, transfected either cell type despite forming complexes with small size and narrow PDI at high N/P ratios. Further, there appeared to be an inverse correlation between the transfection efficacy of the polyplexes and the trend in their zeta potential (FIG. 12). This trend is most noticeable for RAW264.7 macrophages at N/P 20.

### Example 4—Mannosylation of Diblock and Triblock Polymers

To better understand the effect of mannose on the transfection, the ratio of mannosylated to non-mannosylated diblock copolymer was varied and tested in RAW264.7 macrophage transfection. Macrophages RAW264.7, IC21, or BMDMs were analyzed for mannose receptor (MMR; CD206) presence. Samples were analyzed on an LSR II or LSR Fortessa cytometer (BD Biosciences, San Jose, CA). A minimum of 10,000 events were recorded. RAW264.7 cells were chosen as they showed presence of the mannose receptor CD206. Transfection of RAW264.7 cells luciferase expression analysis were performed as described in Example 3. As the ratio of MED2:AED2 increased, the transfection in RAW264.7 cells steadily decreased (FIG. 9B).

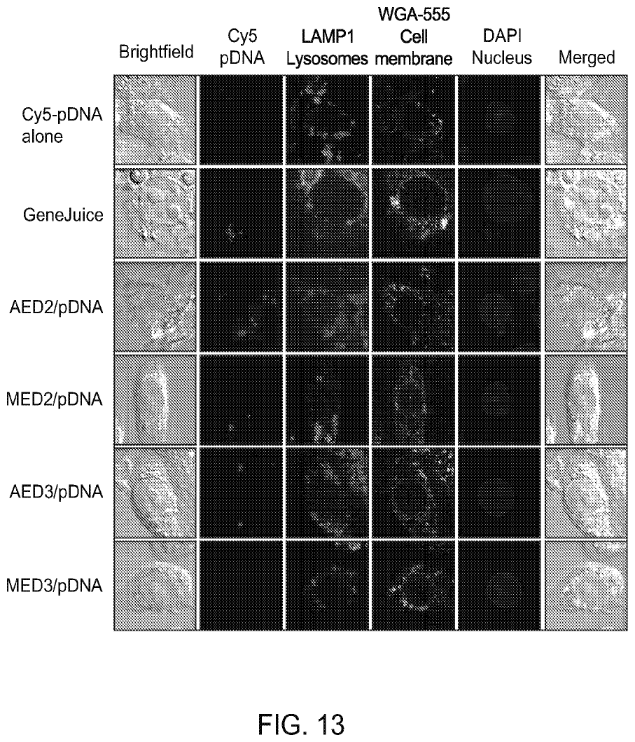

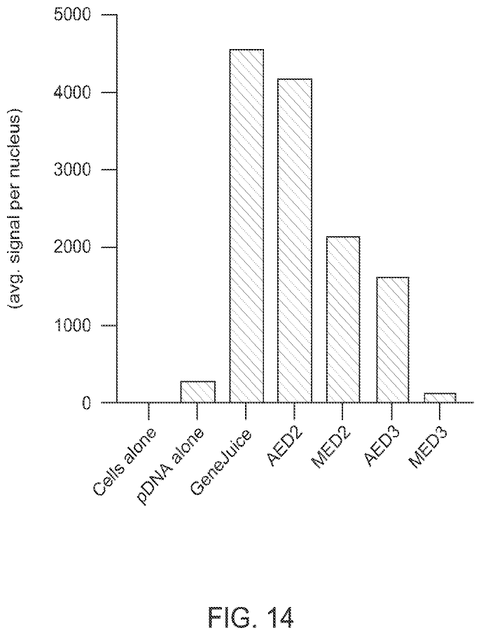

Polyplex uptake and effect of mannose on uptake was further tested using confocal microscopy. In an 8-well Nunc™ Lab-Tek™ II Chambered Coverglass (Thermo Fisher Scientific, Waltham, MA), RAW264.7 macrophages were treated for 24 hours with polyplexes formed at N/P ratio 20 with Cy5-labeled luc-pDNA. The pDNA was covalently labeled with Cy5 using the Label IT™ Nucleic Acid Labeling Kit (Mirus Bio, Madison, WI). Each well was treated with a total of 1 μg Cy5-pDNA. Polyplex uptake was compared to controls such as cells alone, luc-pDNA alone, and GeneJuice transfection reagent. The following cellular compartments were stained: lysosomes (LAMP1), cell membrane (WGA-555), and nuclei (DAPI). Images were taken at 40× magnification on a Zeiss LSM710 (Carl Zeiss AG, Oberkochen, Germany) inverted laser scanning confocal microscope. Confocal imaging revealed that all polyplex formulations, except MED3-based polyplex, at N/P ratio 20 (FIG. 13) were internalized in the cells as shown by clear Cy5 signal in Cy5 channel and merged channel. Polyplexes appear to be localized in or near lysosomes rather than dispersed in the cytoplasm. Cy5 signal was not seen inside the nucleus of any treatment group at this 24-hour timepoint. The confocal imaging data were quantified as an average Cy5 signal intensity per cell nucleus (FIG. 14), which generally agreed with flow cytometry results for quantifying uptake. The results of both quantifications appeared to correlate with the transfection results. The greatest uptake of the luc-pDNA was observed for GeneJuice transfection system and AED2-based polyplexes that also displayed the best transfection results. The uptake of the luc-pDNA in the AED3-based polyplexes was nearly three times less based on the confocal image quantification. Attachment of mannose residues to both diblock and triblock copolymers decreased the uptake of luc-pDNA in each case and was negligible with the MED3-pDNA polyplex, which was also inactive based on the gene expression study.

### Example 5 Polyion Complexes by Dried-Film Method

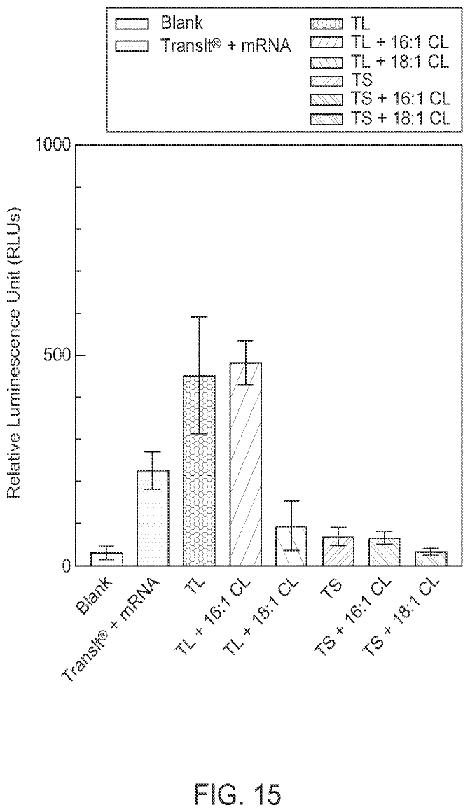

The plot in FIG. 15 shows the result of transfection induced by polyplexes formed by a dried-film method. Luciferase encoding mRNA was included in the polyplexes and HeLa cells were treated with the polyplexes. TransIT® (Mirus Bio, Madison, WI) was used to induce mRNA transfection into cells. Tribloc Long butyl block, p(EtOx)₃₈-p(BuOx)₂₁-p(Mest-DET)₃₁ (TL) showed higher transfection efficiency compared to positive control (TransIT+mRNA). Also, cardiolipin (CL) containing polyplex (TL+16:1 CL) showed a similar extent of transfection efficiency. Shorter triblock short butyl block, p(EtOx)₃₅-p(BuOx)₁₀-p(Mest-DET)₃₀ (TS), still could deliver mRNA into cells but did not lead to higher efficiencies than the positive control. Polyplexes with 18:1 CLs did not induce higher extent of transfection, regardless of polymer. From this data, it was shown that the present system provides better transfection efficiencies than a widely-used positive control (TransIT). It also shows tunability of transfection efficiency with changes in polymer structure.

### Example 6—Freeze-Dried Polyion Complexes

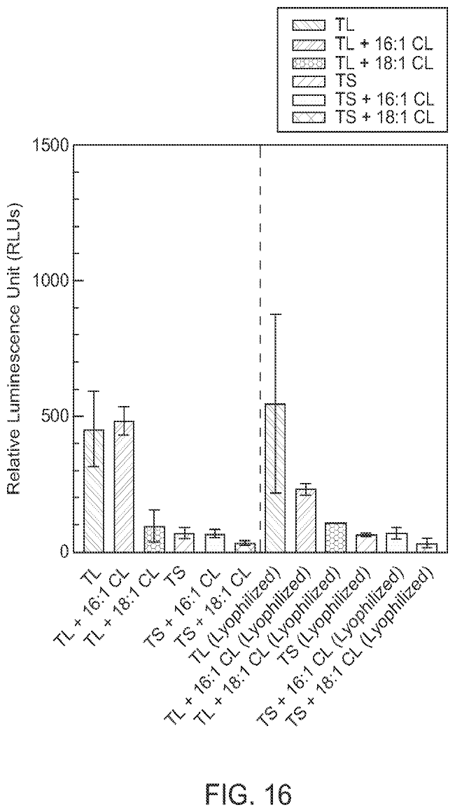

FIG. 16 demonstrates the activity of polyplexes of the invention after a freeze-drying process. Transfection efficiencies of freshly made polyplexes were compared with freeze-dried (and then rehydrated) polyplexes. For polyplexes with TL, significant levels of transfection were shown even after the drying-rehydrating process, though TL with 16:1 CL showed a slightly lower level of transfection efficiency after drying process. It is expected that this process provides a better level of transfer availability than current lipid-based methods.

### Example 7—Polyion Complex Formation in the Presence of Sodium Sulphate

The experiments herein were performed with polymer EBMest-DET (L)-“TL”. Polyplexes were formed via dried film method. The film was prepared by drying 13 μl of solution of TL (5 mg/ml) in EtOH in a 1.6-ml tube under N₂ flow at room temperature for 8 min. Luciferase-encoding unmodified 1929-nt-long mRNA was used. Fifty microliters of mRNA solution 0.2 mg/ml was added to the TL dry film and gently mixed to form the polyplex. These amounts of TL and mRNA corresponded to N/P ratio of 10:1.

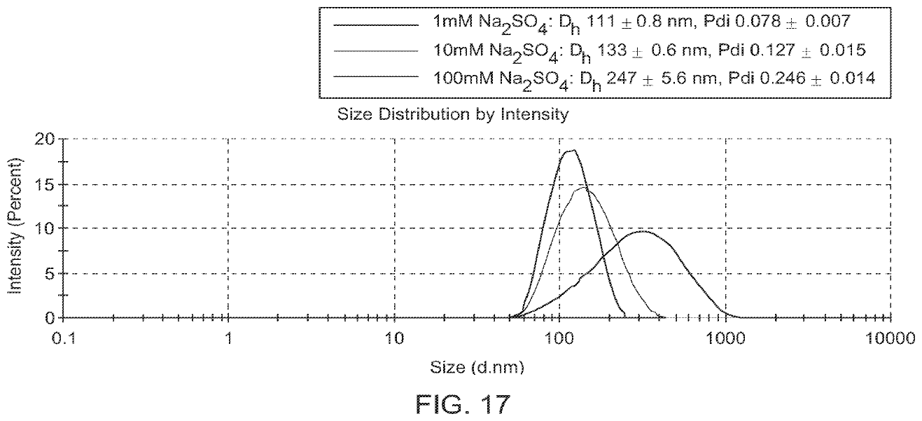

It was found that the presence of sodium sulphate (Na₂SO₄) in the mRNA solution provides favorable conditions for formation of nanoparticles having a narrow peak of their size distribution (FIG. 17). Na₂SO₄ concentration of 1 mM was the optimal one ensuring nanoparticles of about 100 nm in diameter and Pdi<0.1. A reasonable hypothesis on the role of Na₂SO₄ is that the sulphate dianion efficiently interacts with positively charged block of the TL polymer. It shields an excess positive charges of the polymer under conditions of high N/P ratios used for polyplex formation and thus stabilizes the complexes and makes it more compact.

However, polyplexes in Na₂SO₄ alone are unstable: a considerable aggregation of nanoparticles occurs in the timescale of tens on minutes.

### Example 8—Polyion Complex Stabilization by Buffers

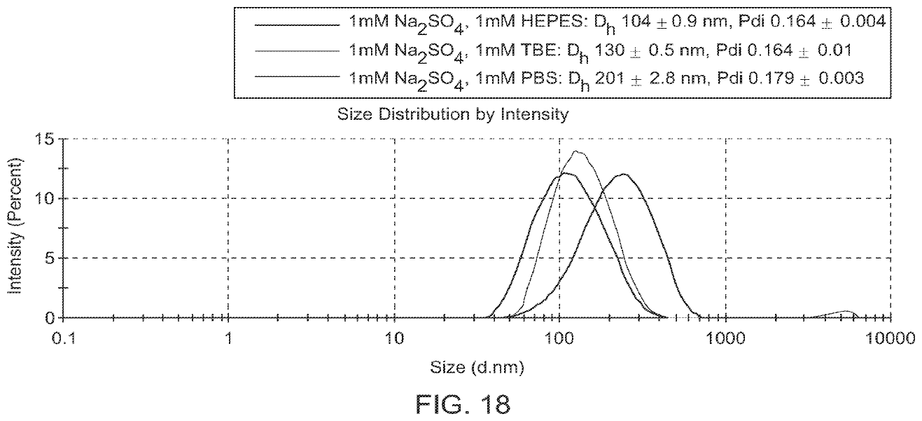

TL/mRNA polyplexes were prepared as described in Example 7. Different buffer solutions were tested: HEPES pH 7.5, TBE pH 8.3, PBS pH 7.0. These buffers were used in combination with 1 mM Na₂SO₄. All buffers were diluted to the final concentration of 1 mM (1 mM HEPES; 1 mM Tris for TBE (i.e., 0.11x standard TBE); 1 mM phosphate buffer for PBS (i.e., 0.08x standard PBS). Polyplexes were formed under conditions described in Example 8 except for adding the buffers to mRNA solutions. HEPES buffer was found to ensure the best characteristics of polyplex nanoparticles, including average diameter of about 100-150 nm and Pdi<0.2 (FIG. 18). Notably, HEPES molecule contains-SO₃ group, which can support interactions with cationic polymer, similarly to sodium sulphate.

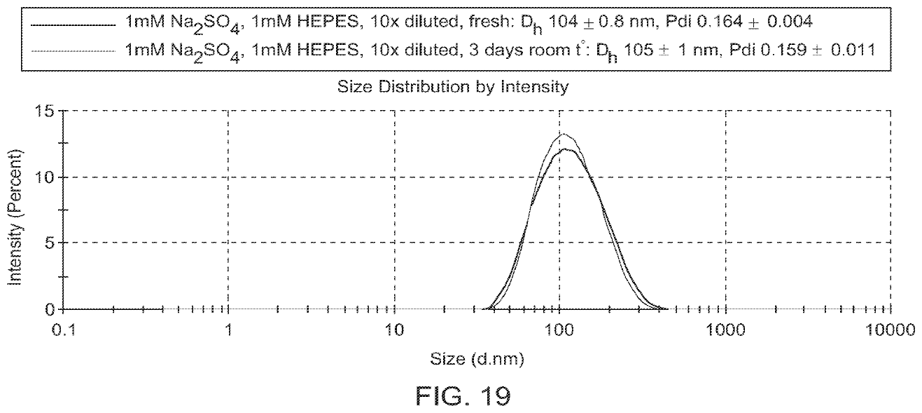

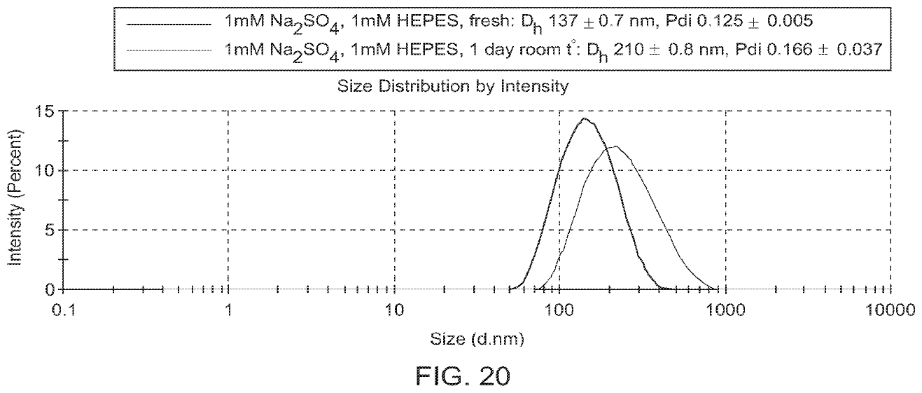

The presence of a buffer greatly increased polyplex stability. In particular, according to DLS data, HEPES ensured full conservation of polyplex structure in the 10-fold diluted solutions (i.e., nanoparticles formed at 0.2 mg/ml mRNA and then diluted to 0.02 mg/ml in the same solution) for at least 3 days at room temperature (FIG. 19). Undiluted samples were stable for at least 1 day at room temperature, with a slightly increased diameter but without aggregation (FIG. 20)

### Example 9—Preparation of Isotonic Solutions with Polyion Complexes

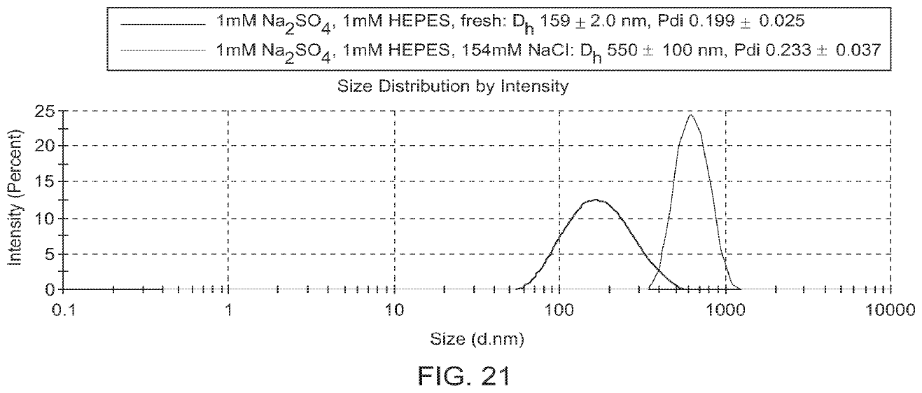

For in vivo injections, polyplex solutions must be isotonic. The most commonly used isotonic solution contains 154 mM (0.9%) NaCl. Thus, influence of 154 mM NaCl to polyplex formation was studied. TL/mRNA polyplexes were prepared as described in Example 7. The mRNA solution (0.2 mg/ml) contained 1 mM Na₂SO₄, 1 mM HEPES. Unfortunately, addition of sodium chloride strongly affected the formation of TL/mRNA polyplexes, making particles much larger and tending to aggregate (FIG. 21). Thus, NaCl cannot be used for preparation of an isotonic solution of polyplexes.

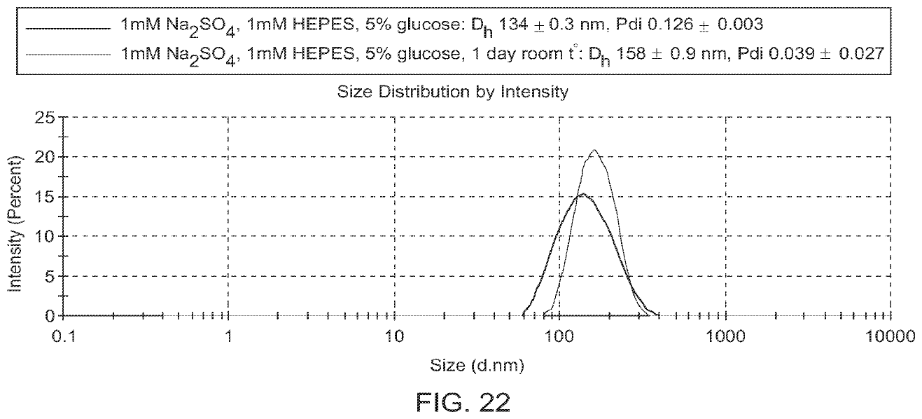

To solve this problem, an alternative isotonic solution, namely 5% glucose (dextrose) was tested. The composition of 1 mM Na₂SO₄, 1 mM HEPES, 5% glucose ensured formation of small and monodisperse TL/mRNA nanoparticles as well as their good stability for at least 1 day in concentrated solutions (0.2 mg/ml mRNA) (FIG. 22). Thus, 5% glucose can be used to compose an isotonic solution for injection of polyplexes in vivo.

### Example 10—Analysis of Polyion Complexes by Gel Electrophoresis

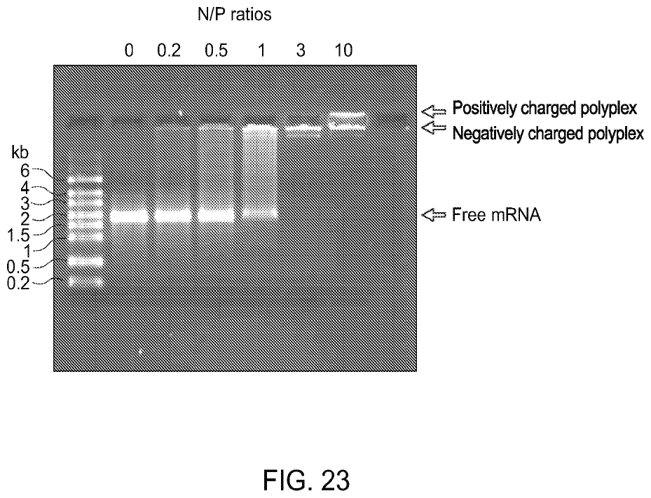

Formation of polyplexes can be analyzed by standard agarose gel electrophoresis similarly to that of DNA and RNA. An example of such analysis is shown in FIG. 23. TL/mRNA polyplexes were prepared as described in Example 7 but different amounts of TL was used to vary N/P ratios (N/P 0.2, 0.5, 1, 3, 10 were tested). The mRNA (0.2 mg/ml) solutions contained 1 mM Na₂SO₄, 1 mM HEPES, 5% glucose.

A standard 1.5% agarose gel with ethidium bromide was prepared and run in 1x TBE. The ethidium bromide staining allows to detect a band of free mRNA of the expected size (1.9 kb) (FIG. 23, light grey arrow). In the presence of the TL polymer, polyplexes are formed, which cannot enter the gel and are detected as a band at the edge of the loading wells (FIG. 23, dark grey arrow). At N/P-3 and above, a complete incorporation of mRNA into polyplex was observed. At N/P=10, a portion of positively charged polyplex is formed (FIG. 23, medium grey arrow).

### Example 11—Formation of Polyion Complexes with N1-Methyl-Pdeudouridine mRNA

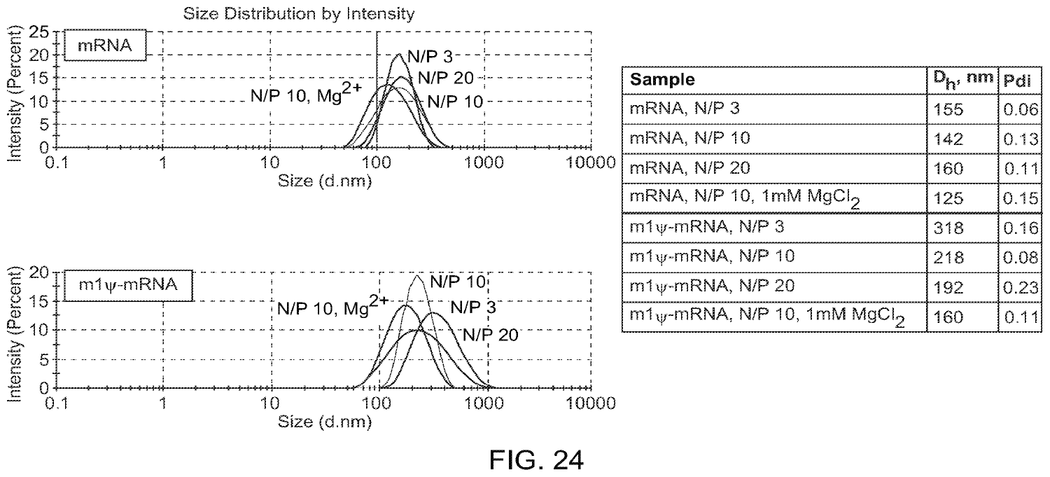

Modified mRNA species containing N1-Methyl-Pdeudouridine (m1Ψ) instead of all uridines are used in all approved mRNA vaccines. The modified mRNA ensures lower nonspecific innate immune response, lower cytotoxicity, higher stability and higher protein synthesis rate. To test polyplex formation with modified mRNA, a luciferase-encoding 2061-nt-long N1-methyl-pseudouridine-mRNA (m14Ψ-mRNA) was used. TL/m1Ψ-mRNA polyplexes were prepared as described in Example 7 except for using the modified mRNA (0.2 mg/ml). The non-modified mRNA (as in the Examples 7-10) was used in some experiments in parallel for comparison. Solution composition selected above (1 mM Na₂SO₄, 1 mM HEPES, 5% glucose) was used. On top of this composition, influence of 1 mM MgCl₂ was also tested, as nucleic acids are known to complex with Mg²⁺ in cells. Different amounts of TL polymer was tested together with a fixed amount of mRNA (0.2 mg/ml) to vary N/P ratio (N/P=3, 10, 20). Undiluted polyplex samples were analyzed by Dynamic Light Scattering (DLS) (FIG. 24). This experiment allows the following conclusion: 1) m1Ψ-mRNA forms larger particles compared to unmodified mRNA. 2) N/P=10 gives the best results for both mRNA and m1Ψ-mRNA among tested N/P ratios. 3) The presence of MgCl₂ results in formation of more compact particles for both mRNA and m1Ψ-mRNA.

Thus, the optimized solution composition for polyplex formation with unmodified and modified N1-Methyl-Pdeudouridine mRNA species is: 1 mM Na₂SO₄, 1 mM HEPES, 1 mM MgCl₂, 5% glucose; TL polymer dry film in amount corresponding to N/P=10.

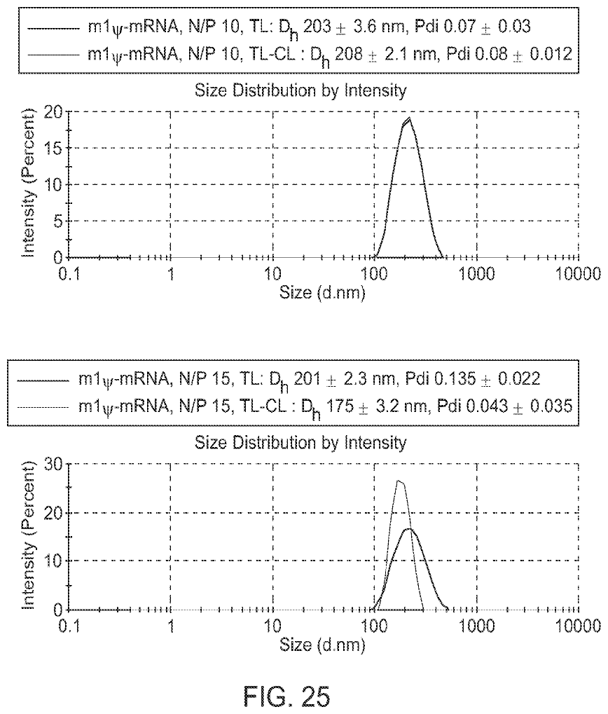

An addition of cardiolipin as an excipient was further tested. TL/m1Ψ-mRNA polyplexes were prepared as described above. The mRNA solution contained 0.2 mg/ml m1Ψ-mRNA, 1 mM Na₂SO₄, 1 mM HEPES, 1 mM MgCl₂, 5% glucose (50 μl per sample). Polymer dry films were prepared using either 5 mg/ml TL in EtOH (“TL” film), or 5 mg/ml TL in EtOH mixed with 1/10 of volume of 5 mg/ml cardiolipin 16:1 in methanol (“TL-CL” film). According to DLS measurements, addition of cardiolipin 16:1 did not affect formation of polyplex nanoparticles (FIG. 25).

### Example 12—Lyophilization of Polyion Complexes

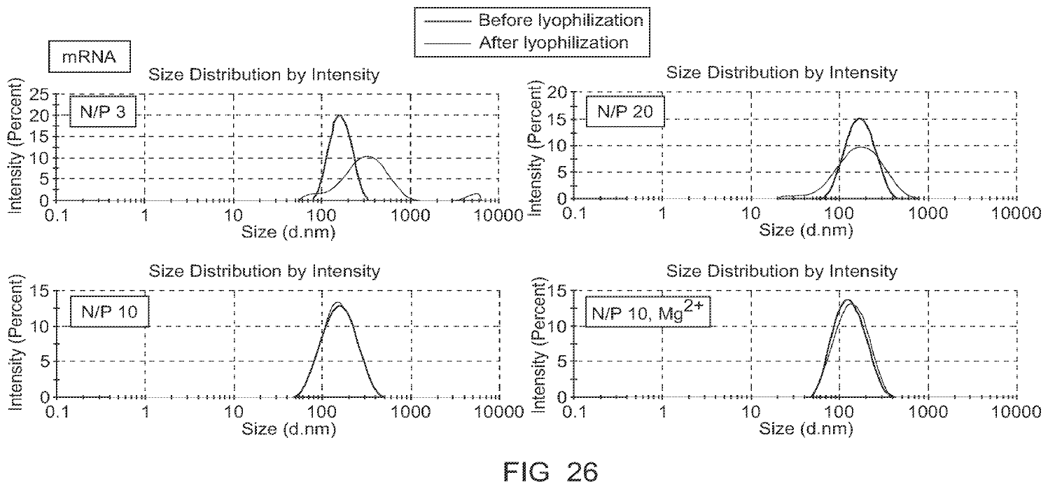

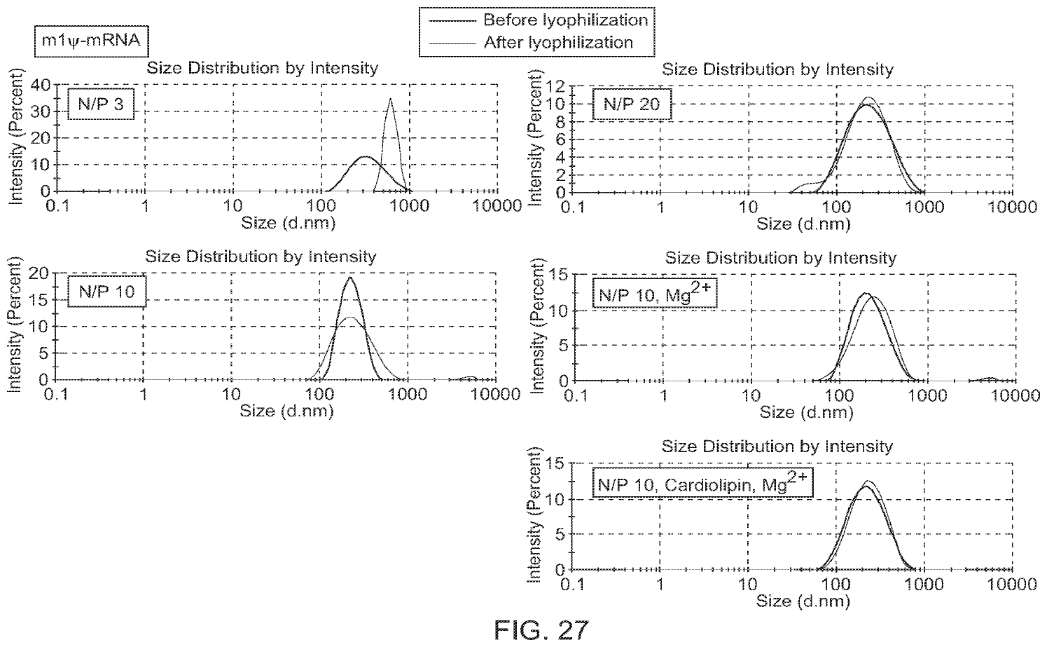

Lyophilization of polyplexes, if successful, provides an efficient, cheap and convenient way of their storage and transportation as lyophilized dry powder can be stored under ambient conditions for a long time. Polyplexes were prepared as described above. Various conditions, namely, different N/P ratios, unmodified or m1Ψ-mRNA species, presence of cardiolipin, were tested. After DLS measurements, the 50-μl samples were frozen in liquid nitrogen and immediately placed into a freeze dryer and dried overnight under vacuum. Next day, the obtained dry powder samples were re-hydrated by addition of 50 μl of water and assessed by DLS (FIGS. 26 and 27).

These experiments allow to conclude the following: 1) both TL/mRNA and TL/m1Ψ-mRNA polyplexes can be successfully lyophilized and re-hydrated; 2) among tested, N/P ratio 10 ensures the best conservation of polyplex structure during lyophilization; 3) presence of 1 mM CaCl₂) is beneficial for m1Ψ-mRNA polyplexes; and 4) presence of cardiolipin 16:1 does not affect lyophilization of m1Ψ-mRNA polyplexes.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention which is set forth in the following claims.

### Example 13—Polyion Complexes Formed Via Dry Films

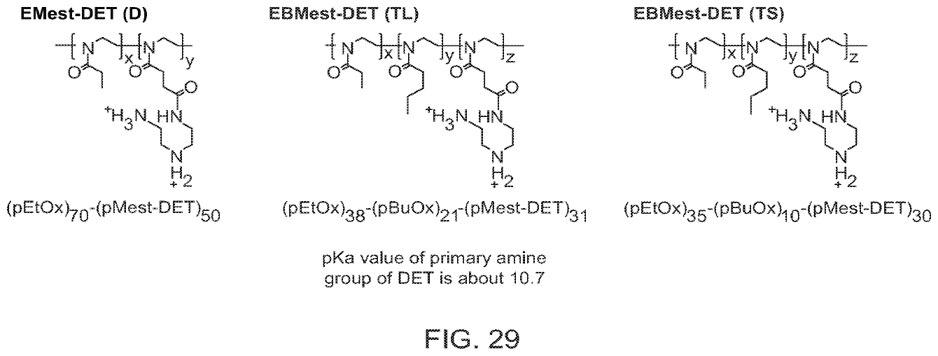

Block copolymer having the structures EMest-DET (D), EBMest-DET (TL), and EBMest-DET (TS) illustrated in FIG. 29 were prepared according to the synthetic procedures described herein. A solution of each polymer was prepares by dissolving the polymer in ethanol at a concentration of 10 mg/mL. To produce the polymeric film, 13 μL of the polymer-ethanol solution was dried under N₂ flow at 60° C. for 10 minutes.

Polyion complexes were subsequently prepared from each of the polymer films (D, TL, TS). An mRNA molecule having 2061 nucleotides was placed in solution with TBE buffer at a concentration of 2 mg/mL. Each dried polymer film was cooled to room temperature and subsequently rehydrated with the mRNA solution. Upon rehydration, the mixture was gently and intermittently vortexed to form polyion complexes.

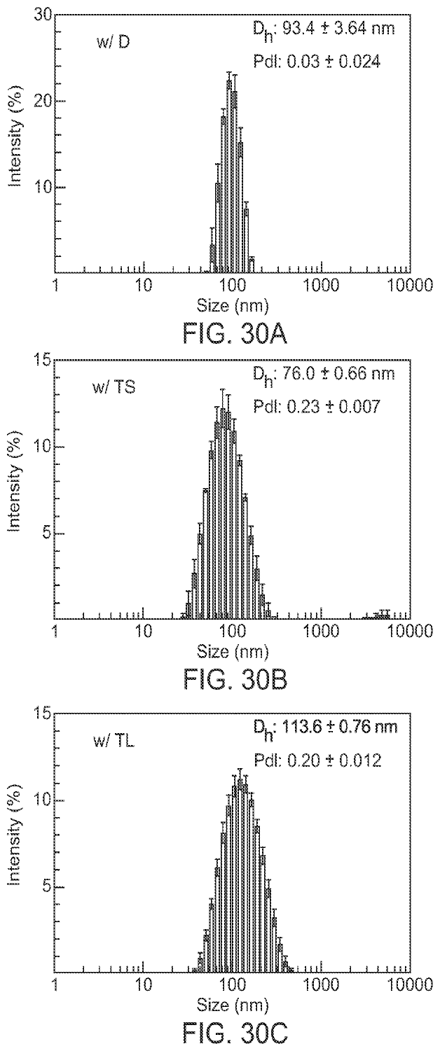

The hydrodynamic diameter of the polyion complexes was controllable upon changes in Pox block length. Diblock copolymer (D) led to polyion complexes having hydrodynamic diameter of about 93 nm (FIG. 30A), tri-block co-polymer containing shorter BuOx block (TS) resulted in the smallest polyplexes (FIG. 30B), and the longer BuOx of tri-block co-polymer (TL) led to the largest hydrodynamic diameter around 113 nm (FIG. 30C).

### Example 14—Polyion Complexes Formed Via Dry Films

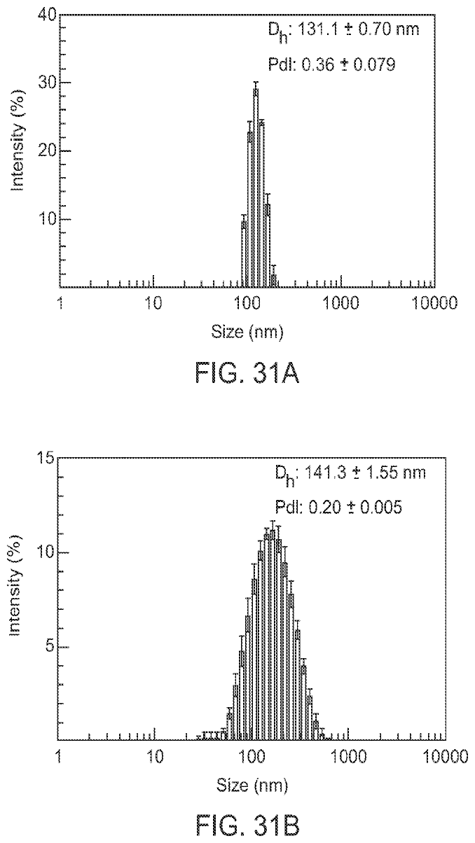

POx-based polyion complexes incorporating block copolymer D were prepared in accordance with the procedure of Example 13. In the present example, two differing mRNA molecules were employed to form the polyion complexes, one mRNA having 3868 nucleotides (FIG. 31A) and a second mRNA having 774 nucleotides (FIG. 31B).

### Example 15—Polyion Complexes Formed Via Dry Films

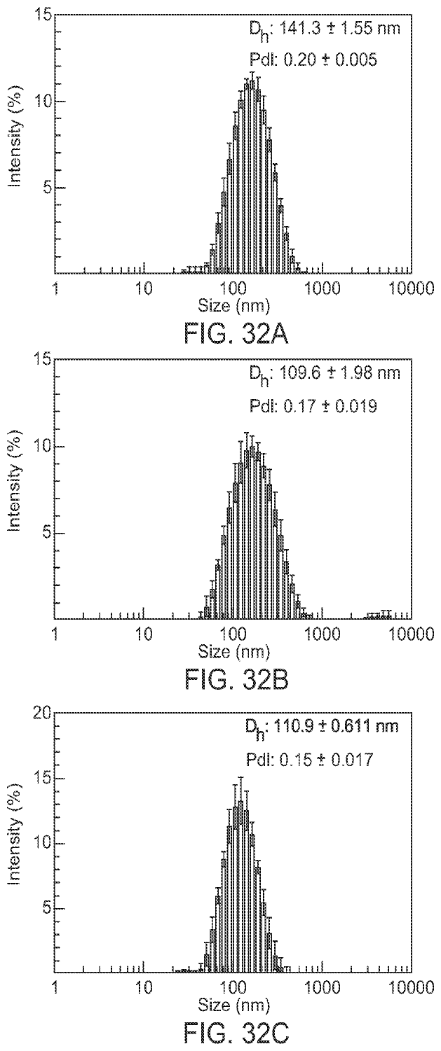

POx-based polyion complexes incorporating block copolymer TL were prepared in accordance with the procedure of Example 13. The mRNA molecule contained 774 nucleotides. In the present example, the block copolymer-ethanol solution contained the addition of excipients cardiolipin 16:1 (FIG. 32B) or cardiolipin 18:1 (FIG. 32C). The polyion complex of FIG. 32A did not employ lipid excipient.

### Example 16—Polyion Complexes Formed Via Dry Films

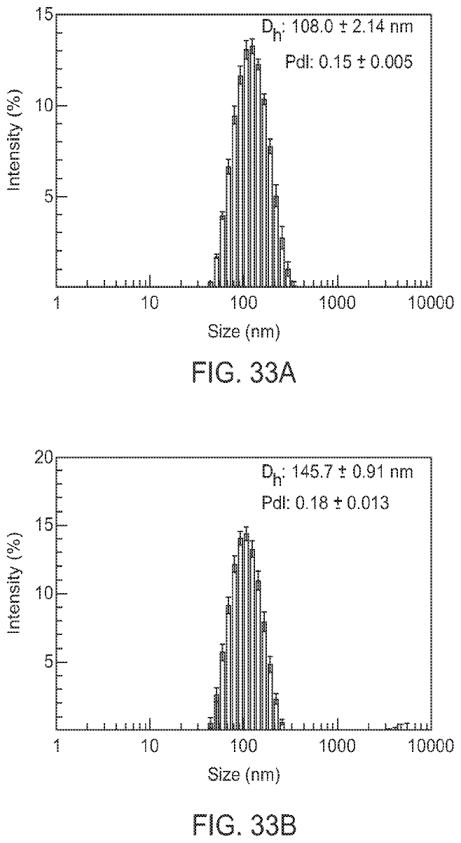

POx-based polyion complexes incorporating block copolymer TL were prepared in accordance with the procedure of Example 13. Structurally modified mRNA complexes were employed in this example, pseudourine-containing mRNA (FIG. 33A) and uridine containing mRNA (FIG. 33B).

### Example 17—Polyion Complexes Formed Via Dry Films

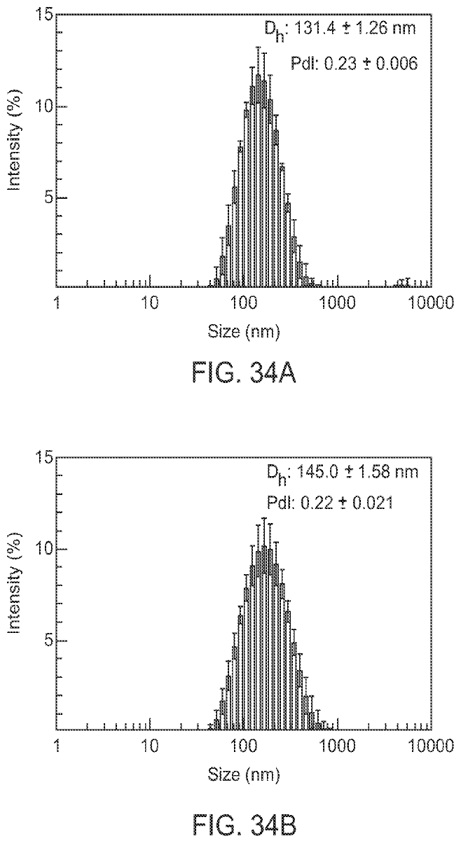

POx-based polyion complexes incorporating block copolymer TL were prepared in accordance with the procedure of Example 13. In the present example, the mRNA solution contained two different mRNA molecules (774 nucleotides and 1929 nucleotides), and the block-copolymer/ethanol solution contained 16:1 cardiolipin excipient (FIG. 34A) and 18:1 cardiolipin excipient (FIG. 34B). As illustrated in FIGS. 34A and 34B, polyion complexes were formed with narrow PDI.

### DISCUSSION

The foregoing Examples are a continued effort in the investigation of poly(2-oxazolines) as a viable alternative to PEG-based transfection polymers. Due to its biocompatibility and ability to conjugate reactive side groups, POx is a promising candidate for replacing PEG in the gene delivery applications. POx has been recently utilized in an assortment of biomedical applications from hydrogels to solubilizing hydrophobic drugs at high capacities.² POx is also less sensitive to oxidative degradation compared to PEG.⁴ Out of the many POx monomers, hydrophilic MeOx and EtOx were employed, with pMeOx being slightly more hydrophilic than pEtOx. Previous findings showed that MeOx-based polymers had lower serum protein binding compared to PEG-based polyplexes, which could result in a longer circulation time in vivo.⁴ Despite the many advantages of POx which lend themselves to being advantageous for transfection, few have developed or characterized such systems for pDNA delivery. The present work seeks to optimize a targeted cationic POx block copolymer for efficient transfection of macrophages which are crucial immune cells in the progression of many cancers such as breast cancer. The design of four polymer block components was tested: non-ionic hydrophilic block, cationic block, hydrophobic block, and targeting moiety. Cationic groups were designed to enhance transfection efficacy and developed a targeting moiety synthesis strategy to enhance uptake via the MMR. The cationic block length was kept at 50 to maintain consistency during comparison of the various polymers. The introduction of a hydrophobic block was also explored to further improve stability and complexation with luc-pDNA for in vivo experiments. Triblock copolymers with i-propyl-2-oxazoline blocks are likely to form structures in which these blocks are segregated in hydrophobic domains. These domains play a role in facilitating nanoparticle formation by additional stabilization through hydrophobic interactions. Furthermore, they can have functional significance by potentially entrapping drugs. One early example of such formations is pDNA complexes with polycation conjugates with Pluronic block copolymers, as are previously described. These complex nanoparticles are sometimes referred to as “micelleplexes” and have been reviewed elsewhere. This study finds that a novel PEG-free POx-based pDNA delivery system is effective at transfecting a variety of macrophages including immortalized cell lines and even primary cells.

Important for designing an optimized system for gene delivery are 1) the chemical composition of the nonionic block, 2) the structure of the cationic block side chains, and 3) the hydrophilicity of the block copolymer, all of which can affect the interaction between polyplexes and cell membranes. When comparing the transfection efficiency of polyplexes made with polymers containing either MeOx or EtOx hydrophilic block, it was found that the EtOx-based polyplexes outperformed those made with most hydrophilic MeOx. Probably, without being beholden to any particular theory, the MeOx shell of the corresponding polyplex was too hydrophilic that it masked not only the binding of the serum proteins as previously shown, but also hindered the polyplex interaction with cells. The effect of the nanoparticle hydrophilic shell structure on their uptake in macrophages has been shown for liposomes coated with PEG and hyperbranched polyglycerol. A previous study found that pMeOx and pEtOx-conjugated protein internalized at higher rates compared to PEGylated protein in CATH.a neuronal cells. This study observed that EtOx-based conjugates are internalized at a ˜4 to ˜7 times faster rate compared to MeOx-based conjugates, which supports why EtOx-based polyplexes transfect cells more efficiently than MeOx-based polyplexes. Since both MeOx and EtOx-based polyplexes formed complexes of similar size, it not likely to be responsible for the difference in IC21 transfection. Overall, the EtOx monomer was chosen for subsequent studies as it showed greatest transfection efficacy.

In the following studies, the focus was on comparing the cationic blocks with different side chains, DET and TREN, to determine which one is more efficient in transfecting macrophages. Previously, DET was used in PEG-containing transfecting polymers. TREN is another cationic moiety which is commonly used in lipid-based transfection systems due to its branched structure which allows for efficient condensation of genetic material. Both cationic moieties, DET and TREN, were chosen based on good biocompatibility, and different charge densities of linear versus branched structures which could impact the complexation with pDNA. TREN-based block copolymers formed tighter complexes perhaps due to the difference in the charge density compared to DET-based block copolymers. Despite forming a more stable complex, TREN-based polyplexes transfected macrophages poorly compared to DET-based polyplexes. As previously reported, tightly bound polyplexes are not able to release their genetic cargo and therefore are worse transfection agents. Notably, the DET-containing copolymers exhibited buffering capacity between pH 5.7 to 7.0, while the TREN-containing copolymers did not. Since the most widely accepted theory of endosomal escape of nucleic acids relies on the ability to attract protons as stated in the proton sponge theory, the DET side chain is a good candidate for nucleic acid delivery into the cell. With both diblock and triblock copolymers, DET proved to be a cationic moiety capable of forming well-defined polyplexes with luc-pDNA leading us to choose it as the optimal cationic block.

Two synthetic click chemistry strategies were used to introduce the targeting moiety to develop the least toxic clickable system for in vivo success. For the cell transfection studies mannose was conjugated via CuAAC rather than copper-free AAC due to a greater mannose conjugation with the CuAAC method. Though the CuAAC method uses copper as a catalyzing reagent, the mannosylated polymers did not show toxicity. Mannosylated copolymers based on DET and EtOx were expected to increase transfection by increasing targeting to macrophages and therefore also increasing uptake. However, both transfection and uptake in macrophages were hindered when using mannosylated polyplexes made from diblock and triblock polymers. Despite a lack of toxicity, and mannose conjugation extent at 37 and 31% for MED2 and MED3 respectively, mannosylation did not improve transfection. Even when varying the ratio of MED2 to non-mannosylated AED2 in polyplex formation, the greater amount of AED2 resulted in increased transfection of RAW264.7 macrophages. Blakney et al. reported that when PEI was modified with mannose, the transfection with small activating RNA (saRNA) in HEK293 cells was decreased, potentially due to steric hindrance of mannose. This group also reported that as amount of mannose moieties attached to PEI was increased, the transfection decreased, which is a similar trend found in the present study. When also comparing EtBr displacement, mannosylated polymers did not displace differently compared to non-mannosylated counterparts meaning that they did not differ much in their binding to pDNA. The localization quantification shows that the cellular uptake of cy5-luc-pDNA is decreased in polyplexes made with mannosylated polymers at N/P ratio 20. Thus, non-mannosylated diblock and triblock polyplexes had greater uptake compared to their mannosylated counterparts. When analyzing the internalization of Cy5-pDNA by flow cytometry, the uptake trend was similar to confocal imaging quantification suggesting that mannosylated polyplex transfection is being hindered during uptake. Mannose may sterically interfere with polyplex uptake, endosomal escape, or pDNA release.

Currently, polyplexes of various sizes are believed to enter the cell through various endocytosis pathways. Notably, the mannosylated copolymer-based polyplexes had similar size by DLS but their uptake in macrophages was inhibited compared to non-targeted polymers. Endocytosis is also governed by shape of particles. The slightly elongated shapes of polyplexes made with mannosylated copolymers could contribute to decreased uptake as Skirtach et al. reports that high-aspect ratio particles result in slower and overall decreased uptake compared to spherical particles due to the forces generated at the interaction between cell and particle. Therefore, the elongated worm-like shape of mannosylated polyplexes can result in decreased uptake, though there is no clear consensus in the literature. Non-mannosylated triblock AED3-based polyplexes also had both low uptake and weak pDNA release which resulted in poor transfection. Since triblock polyplexes had a hydrophobic core, this could cause the formation of complexes which are too stable for releasing pDNA cargo. Therefore, uptake is an indicator of transfection success, and mannosylation on these diblock and triblock copolymers interferes with that process. As mannosylation has been previously reported as an enhancer of internalization, it is surprising that mannose conjugation did not improve uptake or transfection in the present study. The cause of this inhibition may be better understood through studies including flexibility of polymer chains, surface charge at various points during endocytosis, timing of uptake, and incomplete click chemistry.

The present study designed and characterized a POx-based platform for transfecting macrophages with pDNA. Optimal diblock and triblock configurations for highest transfection efficiency consisted of a hydrophilic EtOx block and a cationic DET moiety. The hydrophobic iPrOx block was introduced for the triblock structure which did not improve transfection. The polyplexes exhibited a relatively narrow size distribution and demonstrated safety to macrophages in vitro. Mannosylation of polymers did not enhance the uptake or transfection of macrophages in this specific polymer design. Uptake was also affected by surface charge of complexes where the less positively charged polyplexes transfected the cells more efficiently. Polyplexes made with luc-pDNA and a diblock POx polymer consisting of a hydrophilic EtOx block and a cationic DET moiety transfected both immortalized and primary macrophages with the same efficiency as the commercial transfection reagent, GeneJuice. This study developed an efficient non-toxic PEG-free polymer, AED2, capable of transfecting macrophages with pDNA efficiently.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention which is set forth in the following claims.

1. A block copolymer comprising:
a hydrophilic block comprising oxazoline monomer or oxazine monomer; and
a cationic block comprising monomer including a linear or branched polyamine side chain.

2. The block copolymer of claim 1, wherein the branched polyamine side chain is attached the polymer backbone via an amide linkage.

3. The block copolymer of claim 1, wherein the hydrophilic block is of the formula: 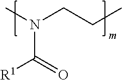
wherein R¹ is selected from the group consisting of alkyl and cycloalkyl, each optionally substituted with hydroxyl, —SH, and C(O)OR², wherein R² is selected from the group consisting of hydrogen and alkyl, and wherein m ranges from 10-300.

4. The block copolymer of claim 1, wherein the polyamine side chain is linear.

5. The block copolymer of claim 1, wherein the polyamine side chain is branched.

6. The block copolymer of claim 1, wherein the cationic block is of the formula: 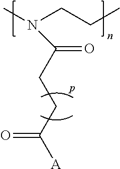
wherein A is the linear or branched or branched polyamine side chain, n ranges from 10-300 and p ranges from 0 to 10.

7. The block copolymer of claim 1, wherein the linear or branched or branched polyamine side chain comprises 3 or more amine functionalities.

8. The block copolymer of claim 1 further comprising a third block being more hydrophobic than the hydrophilic block.

9. The block copolymer of claim 8, wherein the third block comprises oxazoline monomer or oxazine monomer having comprising a propyl or butyl side chain.

10. The block copolymer of claim 1, wherein the block copolymer is terminated in a click chemistry moiety selected from the group consisting of BCN, DBCO, TCO, tetrazine, alkyne, and azide.

11. The block copolymer of claim 10 further comprising a targeting ligand coupled through the click chemistry moiety.

12. The block copolymer of claim 1, wherein the cationic block further comprising non-polyamine monomer.

13. The block copolymer of claim 1, wherein the non-polyamine monomer comprises oxazoline monomer or oxazine monomer having comprising a propyl or butyl side chain.

14. The block copolymer of claim 13, wherein the non-polyamine monomer and monomer including the linear or branched polyamine side chain have a periodic distribution in the cationic block.

15. The block copolymer of claim 13, wherein the non-polyamine monomer and monomer including the linear or branched polyamine side chain have a random distribution in the cationic block.

16. A polyion complex comprising:
a block copolymer comprising a hydrophilic block comprising oxazoline monomer or oxazine monomer, and a cationic block comprising monomer including a linear or branched cationic polyamine side chain; and
a negatively charged biomolecular species associated with the block copolymer.

17. The polyion complex of claim 16, wherein the negatively charged biomolecular species comprises one or more nucleic acids.

18. The polyion complex of claim 17, wherein the biomolecular species is RNA or DNA.

19. The polyion complex of claim 18, wherein the RNA is selected from the group consisting of messenger RNA (mRNA), transfer RNA (RNA), ribosomal RNA (rRNA), and small-activating RNA (sa-RNA).

20. The polyion complex of claim 16, wherein the hydrophilic block is of the formula: 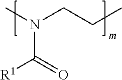
wherein R¹ is selected from the group consisting of alkyl and cycloalkyl, each optionally substituted with hydroxyl, —SH, and C(O)OR², wherein R² is selected from the group consisting of hydrogen and alkyl, and wherein m ranges from 10-300.

21. The polyion complex of claim 16, wherein the cationic block is of the formula: 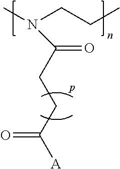
wherein A is the linear or branched or branched polyamine side chain, n ranges from 10-300 and p ranges from 0 to 10.

22. The polyion complex of claim 16, wherein the linear or branched or branched polyamine side chain comprises 3 or more amine functionalities.

23. The polyion complex of claim 16, wherein the block copolymer further comprises a third block being more hydrophobic than the hydrophilic block.

24. The polyion complex of claim 23, wherein the third block comprises oxazoline monomer or oxazine monomer having comprising a propyl or butyl side chain.

25. The polyion complex of claim 16, wherein the cationic block further comprising non-polyamine monomer.

26. The polyion complex of claim 25, wherein the non-polyamine monomer comprises oxazoline monomer or oxazine monomer having comprising a propyl or butyl side chain.

27. The polyion complex of claim 26, wherein the non-polyamine monomer and monomer including the linear or branched polyamine side chain have a periodic distribution in the cationic block.

28. The polyion complex of claim 26, wherein the non-polyamine monomer and monomer including the linear or branched polyamine side chain have a random distribution in the cationic block.

29. The polyion complex of claim 16 having a hydrodynamic diameter of 50 nm to 150 nm.

30. A dispersion comprising:
an aqueous or aqueous-based continuous phase; and
a dispersed phase comprising polyion complexes, the polyion complexes comprising a block copolymer including a hydrophilic block comprising oxazoline monomer or oxazine monomer, and a cationic block comprising monomer including a linear or branched cationic polyamine side chain, and a negatively charged biomolecular species associated with the block copolymer.

31. The dispersion of claim 30, wherein the negatively charged biomolecular species comprises one or more nucleic acids.

32. The dispersion of claim 31, wherein the biomolecular species is RNA.

33. The dispersion of claim 32, wherein the RNA is selected from the group consisting of messenger RNA (mRNA), transfer RNA (RNA), ribosomal RNA (rRNA), and small-activating RNA (sa-RNA).

34. The dispersion of claim 31, wherein the biomolecular species is DNA.

35. The dispersion of claim 30, wherein the hydrophilic block is of the formula: 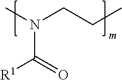
wherein R¹ is selected from the group consisting of alkyl and cycloalkyl, each optionally substituted with hydroxyl, —SH, and C(O)OR², wherein R² is selected from the group consisting of hydrogen and alkyl, and wherein m ranges from 10-300.

36. The dispersion of claim 30, wherein the cationic block is of the formula: 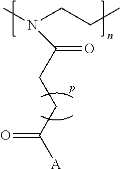
wherein A is the linear or branched or branched polyamine side chain, n ranges from 10-300 and p ranges from 0 to 10.

37. The dispersion of claim 30, wherein the block copolymer further comprises a third block being more hydrophobic than the hydrophilic block.

38-72. (canceled)
